SEQUENCE LISTING (346 sequences)

SEQ ID NO: 1 - protein, 1246 aa, Unknown
  Description of Unknown Lachnospiraceae bacterium ND2006 sequence
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1227 aa, Unknown
  Description of Unknown Candidatus Methanomethylophilus alvus Mx1201 sequence
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 3 - protein, 999 aa, Sneathia amnii
MNLLINEVNQKYKSKLNVFNSLYKQILTEEDTKSFVDEILLTPESVIKTIDNFIDSIIMNDIEGLKEEFLKISLENFEGIYISNKKLNEISNRKFGDYNSINMMIKQSMNEKGILSKKEINELIPDLENINKPKVKSFNLSFIFENLTKEHKELIIDYIRENICNVIENVKITIEKYRNIDNKIEFKNNAEKVSKIKEMLESINELCKLIKEFNTDEIEKNNEFYNILNKNFEIFESSYKVLNKVRNFVTKKEVIENKMKLNFSNYQLGNGWHKNKEKDCSIILFRKRNNERWIYYLGILKHGTKIKENDYLSSVDTGFYKMDYYAQNSLSKMIPKCSITVKNVKNAPEDESVILNDSKKFNEPLEITPEIRKLYGNNEHIKGDKFKKESLVKWIDFCKEFLLKYKSFEKAKKEILKLKESNLYENLEEFYSDAEEKAYFLEFINIDEDKIKKLVKEKNLYLFQIYNKDFSAYSTGNKNLHTMYFEELFTDENLKKPVFKLNGNTEVFYRIASSKPKIVHNKGEKLVNKTYLDDGIIKTIPDSVYEEISEKVKNNEDYSKLLEENNIKNLEIKVATHEIVKDKRYFENKFLFYLPITLNKKVSNKNTNKNINKNVIDEIKDCNEYNVIGIDRGERNLISLCIINQNGEIILQKEMNIIQSSDKYNVDYNEKLEIKSKERDNAKKNWSEIGKIKDLKSGYLSAVVHEIVKLAIEYNAVIILEDLNNGFKNSRKKVDKQIYQKFERALIEKLQFLIFKNYDKNEKGGLRNAFQLTPELKNITKVASQQGIIIYTNPAYTSKIDPTTGYANIIKKSNNNEESIVKAIDKISYDKEKDMFYFDINLSNSSFNLTVKNVLKKEWRIYTNGERIIYKDRKYITLNITQEMKDILSKCGIDYLNIDNLKQDILKNKLHKKVYYIFELANKMRNENKDVDYIISPVLNKDGKFFMTQEINELTPKDADLNGAYNIALKGKLMIDNLNKKEKFVFLSNEDWLNFIQGR

SEQ ID NO: 4 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 5 - protein, 1331 aa, Unknown
  Description of Unknown Parcubacteria group bacterium GW2011_GWF2_44_17 sequence
MKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 6 - protein, 1285 aa, Unknown
  Description of Unknown Candidatus Roizmanbacteria bacterium GW2011_GWA2_37_7 sequence
MKSFDSFTNLYSLSKTLKFEMRPVGNTQKMLDNAGVFEKDKLIQKKYGKTKPYFDRLHREFIEEALTGVELIGLDENFRTLVDWQKDKKNNVAMKAYENSLQRLRTEIGKIFNLKAEDWVKNKYPILGLKNKNTDILFEEAVFGILKARYGEEKDTFIEVEEIDKTGKSKINQISIFDSWKGFTGYFKKFFETRKNFYKNDGTSTAIATRIIDQNLKRFIDNLSIVESVRQKVDLAETEKSFSISLSQFFSIDFYNKCLLQDGIDYYNKIIGGETLKNGEKLIGLNELINQYRQNNKDQKIPFFKLLDKQILSEKILFLDEIKNDTELIEALSQFAKTAEEKTKIVKKLFADFVENNSKYDLAQIYISQEAFNTISNKWTSETETFAKYLFEAMKSGKLAKYEKKDNSYKFPDFIALSQMKSALLSISLEGHFWKEKYYKISKFQEKTNWEQFLAIFLYEFNSLFSDKINTKDGETKQVGYYLFAKDLHNLILSEQIDIPKDSKVTIKDFADSVLTIYQMAKYFAVEKKRAWLAEYELDSFYTQPDTGYLQFYDNAYEDIVQVYNKLRNYLTKKPYSEEKWKLNFENSTLANGWDKNKESDNSAVILQKGGKYYLGLITKGHNKIFDDRFQEKFIVGIEGGKYEKIVYKFFPDQAKMFPKVCFSAKGLEFFRPSEEILRIYNNAEFKKGETYSIDSMQKLIDFYKDCLTKYEGWACYTFRHLKPTEEYQNNIGEFFRDVAEDGYRIDFQGISDQYIHEKNEKGELHLFEIHNKDWNLDKARDGKSKTTQKNLHTLYFESLFSNDNVVQNFPIKLNGQAEIFYRPKTEKDKLESKKDKKGNKVIDHKRYSENKIFFHVPLTLNRTKNDSYRFNAQINNFLANNKDINIIGVDRGEKHLVYYSVITQASDILESGSLNELNGVNYAEKLGKKAENREQARRDWQDVQGIKDLKKGYISQVVRKLADLAIKHNAIIILEDLNMRFKQVRGGIEKSIYQQLEKALIDKLSFLVDKGEKNPEQAGHLLKAYQLSAPFETFQKMGKQTGIIFYTQASYTSKSDPVTGWRPHLYLKYFSAKKAKDDIAKFTKIEFVNDRFELTYDIKDFQQAKEYPNKTVWKVCSNVERFRWDKNLNQNKGGYTHYTNITENIQELFTKYGIDITKDLLTQISTIDEKQNTSFFRDFIFYFNLICQIRNTDDSEIAKKNGKDDFILSPVEPFFDSRKDNGNKLPENGDDNGAYNIARKGIVILNKISQYSEKNENCEKMKWGDLYVSNIDWDNFVTQANARH

SEQ ID NO: 7 - protein, 1477 aa, Unknown
  Description of Unknown Candidatus Peregrinibacteria bacterium GW2011_GWC2_33_13 sequence
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 8 - protein, 1206 aa, Unknown
  Description of Unknown Lachnospiraceae bacterium MA2020 sequence
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 9 - protein, 1206 aa, Butyrivibrio sp.
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 10 - protein, 1231 aa, Butyrivibrio fibrisolvens
MYYESLTKLYPIKKTIRNELVPIGKTLENIKKNNILEADEDRKIAYIRVKAIMDDYHKRLINEALSGFALIDLDKAANLYLSRSKSADDIESFSRFQDKLRKAIAKRLREHENFGKIGNKDIIPLLQKLSENEDDYNALESFKNFYTYFESYNDVRLNLYSDKEKSSTVAYRLINENLPRFLDNIRAYDAVQKAGITSEELSSEAQDGLFLVNTFNNVLIQDGINTYNEDIGKLNVAINLYNQKNASVQGFRKVPKMKVLYKQILSDREESFIDEFESDTELLDSLESHYANLAKYFGSNKVQLLFTALRESKGVNVYVKNDIAKTSFSNVVFGSWSRIDELINGEYDDNNNRKKDEKYYDKRQKELKKNKSYTIEKIITLSTEDVDVIGKYIEKLESDIDDIRFKGKNFYEAVLCGHDRSKKLSKNKGAVEAIKGYLDSVKDFERDLKLINGSGQELEKNLVVYGEQEAVLSELSGIDSLYNMTRNYLTKKPFSTEKIKLNFNKPTFLDGWDYGNEEAYLGFFMIKEGNYFLAVMDANWNKEFRNIPSVDKSDCYKKVIYKQISSPEKSIQNLMVIDGKTVKKNGRKEKEGIHSGENLILEELKNTYLPKKINDIRKRRSYLNGDTFSKKDLTEFIGYYKQRVIEYYNGYSFYFKSDDDYASFKEFQEDVGRQAYQISYVDVPVSFVDDLINSGKLYLFRVYNKDFSEYSKGRLNLHTLYFKMLFDERNLKNVVYKLNGQAEVFYRPSSIKKEELIVHRAGEEIKNKNPKRAAQKPTRRLDYDIVKDRRYSQDKFMLHTSIIMNFGAEENVSFNDIVNGVLRNEDKVNVIGIDRGERNLLYVVVIDPEGKILEQRSLNCITDSNLDIETDYHRLLDEKESDRKIARRDWTTIENIKELKAGYLSQVVHIVAELVLKYNAIICLEDLNFGFKRGRQKVEKQVYQKFEKMLIDKLNYLVMDKSREQLSPEKISGALNALQLTPDFKSFKVLGKQTGIIYYVPAYLTSKIDPMTGFANLFYVKYENVDKAKEFFSKFDSIKYNKDGKNWNTKGYFEFAFDYKKFTDRAYGRVSEWTVCTVGERIIKFKNKEKNNSYDDKVIDLTNSLKELFDSYKVTYESEVDLKDAILAIDDPAFYRDLTRRLQQTLQMRNSSCDGSRDYIISPVKNSKGEFFCSDNNDDTTPNDADANGAFNIARKGLWVLNEIRNSEEGSKINLAMSNAQWLEYAQDNTI

SEQ ID NO: 11 - protein, 1247 aa, Prevotella bryantii
MKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 12 - protein, 1262 aa, Unknown
  Description of Unknown Bacteroidetes oral taxon 274 str. F0058 sequence
MRKFNEFVGLYPISKTLRFELKPIGKTLEHIQRNKLLEHDAVRADDYVKVKKIIDKYHKCLIDEALSGFTFDTEADGRSNNSLSEYYLYYNLKKRNEQEQKTFKTIQNNLRKQIVNKLTQSEKYKRIDKKELITTDLPDFLTNESEKELVEKFKNFTTYFTEFHKNRKNMYSKEEKSTAIAFRLINENLPKFVDNIAAFEKVVSSPLAEKINALYEDFKEYLNVEEISRVFRLDYYDELLTQKQIDLYNAIVGGRTEEDNKIQIKGLNQYINEYNQQQTDRSNRLPKLKPLYKQILSDRESVSWLPPKFDSDKNLLIKIKECYDALSEKEKVFDKLESILKSLSTYDLSKIYISNDSQLSYISQKMFGRWDIISKAIREDCAKRNPQKSRESLEKFAERIDKKLKTIDSISIGDVDECLAQLGETYVKRVEDYFVAMGESEIDDEQTDTTSFKKNIEGAYESVKELLNNADNITDNNLMQDKGNVEKIKTLLDAIKDLQRFIKPLLGKGDEADKDGVFYGEFTSLWTKLDQVTPLYNMVRNYLTSKPYSTKKIKLNFENSTLMDGWDLNKEPDNTTVIFCKDGLYYLGIMGKKYNRVFVDREDLPHDGECYDKMEYKLLPGANKMLPKVFFSETGIQRFLPSEELLGKYERGTHKKGAGFDLGDCRALIDFFKKSIERHDDWKKFDFKFSDTSTYQDISEFYREVEQQGYKMSFRKVSVDYIKSLVEEGKLYLFQIYNKDFSAHSKGTPNMHTLYWKMLFDEENLKDVVYKLNGEAEVFFRKSSITVQSPTHPANSPIKNKNKDNQKKESKFEYDLIKDRRYTVDKFLFHVPITMNFKSVGGSNINQLVKRHIRSATDLHIIGIDRGERHLLYLTVIDSRGNIKEQFSLNEIVNEYNGNTYRTDYHELLDTREGERTEARRNWQTIQNIRELKEGYLSQVIHKISELAIKYNAVIVLEDLNFGFMRSRQKVEKQVYQKFEKMLIDKLNYLVDKKKPVAETGGLLRAYQLTGEFESFKTLGKQSGILFYVPAWNTSKIDPVTGFVNLFDTHYENIEKAKVFFDKFKSIRYNSDKDWFEFVVDDYTRFSPKAEGTRRDWTICTQGKRIQICRNHQRNNEWEGQEIDLTKAFKEHFEAYGVDISKDLREQINTQNKKEFFEELLRLLRLTLQMRNSMPSSDIDYLISPVANDTGCFFDSRKQAELKENAVLPMNADANGAYNIARKGLLAIRKMKQEENDSAKISLAISNKEWLKFAQTKPYLED

SEQ ID NO: 13 - protein, 1318 aa, Flavobacterium branchiophilum
MTNKFTNQYSLSKTLRFELIPQGKTLEFIQEKGLLSQDKQRAESYQEMKKTIDKFHKYFIDLALSNAKLTHLETYLELYNKSAETKKEQKFKDDLKKVQDNLRKEIVKSFSDGDAKSIFAILDKKELITVELEKWFENNEQKDIYFDEKFKTFTTYFTGFHQNRKNMYSVEPNSTAIAYRLIHENLPKFLENAKAFEKIKQVESLQVNFRELMGEFGDEGLIFVNELEEMFQINYYNDVLSQNGITIYNSIISGFTKNDIKYKGLNEYINNYNQTKDKKDRLPKLKQLYKQILSDRISLSFLPDAFTDGKQVLKAIFDFYKINLLSYTIEGQEESQNLLLLIRQTIENLSSFDTQKIYLKNDTHLTTISQQVFGDFSVFSTALNYWYETKVNPKFETEYSKANEKKREILDKAKAVFTKQDYFSIAFLQEVLSEYILTLDHTSDIVKKHSSNCIADYFKNHFVAKKENETDKTFDFIANITAKYQCIQGILENADQYEDELKQDQKLIDNLKFFLDAILELLHFIKPLHLKSESITEKDTAFYDVFENYYEALSLLTPLYNMVRNYVTQKPYSTEKIKLNFENAQLLNGWDANKEGDYLTTILKKDGNYFLAIMDKKHNKAFQKFPEGKENYEKMVYKLLPGVNKMLPKVFFSNKNIAYFNPSKELLENYKKETHKKGDTFNLEHCHTLIDFFKDSLNKHEDWKYFDFQFSETKSYQDLSGFYREVEHQGYKINFKNIDSEYIDGLVNEGKLFLFQIYSKDFSPFSKGKPNMHTLYWKALFEEQNLQNVIYKLNGQAEIFFRKASIKPKNIILHKKKIKIAKKHFIDKKTKTSEIVPVQTIKNLNMYYQGKISEKELTQDDLRYIDNFSIFNEKNKTIDIIKDKRFTVDKFQFHVPITMNFKATGGSYINQTVLEYLQNNPEVKIIGLDRGERHLVYLTLIDQQGNILKQESLNTITDSKISTPYHKLLDNKENERDLARKNWGTVENIKELKEGYISQVVHKIATLMLEENAIVVMEDLNFGFKRGRFKVEKQIYQKLEKMLIDKLNYLVLKDKQPQELGGLYNALQLTNKFESFQKMGKQSGFLFYVPAWNTSKIDPTTGFVNYFYTKYENVDKAKAFFEKFEAIRFNAEKKYFEFEVKKYSDFNPKAEGTQQAWTICTYGERIETKRQKDQNNKFVSTPINLTEKIEDFLGKNQIVYGDGNCIKSQIASKDDKAFFETLLYWFKMTLQMRNSETRTDIDYLISPVMNDNGTFYNSRDYEKLENPTLPKDADANGAYHIAKKGLMLLNKIDQADLTKKVDLSISNRDWLQFVQKNK

SEQ ID NO: 14 - protein, 1235 aa, Unknown
  Description of Unknown Lachnospiraceae bacterium MC2017 sequence
MTMDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKLLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 15 - protein, 1251 aa, Moraxella lacunata
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGPNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 16 - protein, 1251 aa, Moraxella bovoculi
MLFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 17 - protein, 1261 aa, Moraxella bovoculi
MLFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 18 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 19 - protein, 1298 aa, Thiomicrospira sp.
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 20 - RNA, 20 nt, Artificial Sequence
  Description of Artificial Sequence Synthetic oligonucleotide
uggccuggaggcuauccagc

SEQ ID NO: 21 - RNA, 20 nt, Artificial Sequence
  Description of Artificial Sequence Synthetic oligonucleotide
auauaaguggaggcgucgcg

SEQ ID NO: 22 - RNA, 20 nt, Artificial Sequence
  Description of Artificial Sequence Synthetic oligonucleotide
cucacgucauccagcagaga

SEQ ID NO: 23 - RNA, 23 nt, Artificial Sequence
  Description of Artificial Sequence Synthetic oligonucleotide
auuaaauaguagcugagcugauc

SEQ ID NO: 24 - RNA, 23 nt, Artificial Sequence
  Description of Artificial Sequence Synthetic oligonucleotide
gcacuacuaaagucaguggggac

SEQ ID NO: 25 - RNA, 23 nt, Artificial Sequence
  Description of Artificial Sequence Synthetic oligonucleotide
guaaugacacugucgucauucca

SEQ ID NO: 26 - RNA, 23 nt, Artificial Sequence
  Description of Artificial Sequence Synthetic oligonucleotide
agcagggcuuccucuggagcuuc

SEQ ID NO: 27 - RNA, 23 nt, Artificial Sequence
  Description of Artificial Sequence Synthetic oligonucleotide
uuguggugcgcguguuccaaggc

SEQ ID NO: 28 - RNA, 23 nt, Artificial Sequence
  Description of Artificial Sequence Synthetic oligonucleotide
cuggccgccuucugcuggaugag

SEQ ID NO: 29 - RNA, 20 nt, Artificial Sequence
  Description of Artificial Sequence Synthetic oligonucleotide
ucaugcuugcuugggcaaaa

SEQ ID NO: 30 - RNA, 19 nt, Artificial Sequence
  Description of Artificial Sequence Synthetic oligonucleotide
aauuucuacucuuguagau

SEQ ID NO: 31 - RNA, 19 nt, Artificial Sequence
  Description of Artificial Sequence Synthetic oligonucleotide
gauuucuacucuuguagau

SEQ ID NO: 32 - RNA, 19 nt, Artificial Sequence
  Description of Artificial Sequence Synthetic oligonucleotide
uuaaucuacucuuguagau

SEQ ID NO: 33 - RNA, 19 nt, Artificial Sequence
  Description of Artificial Sequence Synthetic oligonucleotide
gugacgacggcugugggcc

SEQ ID NO: 34 - RNA, 20 nt, Artificial Sequence
  Description of Artificial Sequence Synthetic oligonucleotide
aauuucuacuaaguguagau

SEQ ID NO: 35 - RNA, 19 nt, Artificial Sequence
  Description of Artificial Sequence Synthetic oligonucleotide
aauuucuacuaguguagau

SEQ ID NO: 36 - RNA, 19 nt, Artificial Sequence
  Description of Artificial Sequence Synthetic oligonucleotide
aauuucuacuauuguagau

SEQ ID NO: 37 - RNA, 19 nt, Artificial Sequence
  Description of Artificial Sequence Synthetic oligonucleotide
aauuucuacucuuguagau

SEQ ID NO: 38 - RNA, 19 nt, Artificial Sequence
  Description of Artificial Sequence Synthetic oligonucleotide
aauuucuacuuuuguagau

SEQ ID NO: 39 - RNA, 19 nt, Artificial Sequence
  Description of Artificial Sequence Synthetic oligonucleotide
aauuucuacuuuuguagau

SEQ ID NO: 40 - RNA, 19 nt, Artificial Sequence
  Description of Artificial Sequence Synthetic oligonucleotide
gauuucuacuuuuguagau

SEQ ID NO: 41 - RNA, 19 nt, Artificial Sequence
  Description of Artificial Sequence Synthetic oligonucleotide
aauuucuacuauuguagau

SEQ ID NO: 42 - RNA, 19 nt, Artificial Sequence
  Description of Artificial Sequence Synthetic oligonucleotide
aauuucuacuauuguagau

SEQ ID NO: 43 - RNA, 19 nt, Artificial Sequence
  Description of Artificial Sequence Synthetic oligonucleotide
aauuucuacuguuguagau

SEQ ID NO: 44 - RNA, 19 nt, Artificial Sequence
  Description of Artificial Sequence Synthetic oligonucleotide
aauuucuacuauuguagau

SEQ ID NO: 45 - RNA, 19 nt, Artificial Sequence
  Description of Artificial Sequence Synthetic oligonucleotide
aauuucuacuauuguagau

SEQ ID NO: 46 - RNA, 19 nt, Artificial Sequence
  Description of Artificial Sequence Synthetic oligonucleotide
aauuucuacuauuguagau

SEQ ID NO: 47 - RNA, 19 nt, Artificial Sequence
  Description of Artificial Sequence Synthetic oligonucleotide
aauuucuacuguuguagau

SEQ ID NO: 48 - RNA, 20 nt, Artificial Sequence
  Description of Artificial Sequence Synthetic oligonucleotide
aauuucuacuguuuguagau

SEQ ID NO: 49 - RNA, 20 nt, Artificial Sequence
  Description of Artificial Sequence Synthetic oligonucleotide
aauuucuacuguuuguagau

SEQ ID NO: 50 - RNA, 20 nt, Artificial Sequence
  Description of Artificial Sequence Synthetic oligonucleotide
aauuucuacuguuuguagau

SEQ ID NO: 51 - RNA, 19 nt, Artificial Sequence
  Description of Artificial Sequence Synthetic oligonucleotide
aauuucuacuguuguagau

SEQ ID NO: 52 - RNA, 19 nt, Artificial Sequence
  Description of Artificial Sequence Synthetic oligonucleotide
aauuucuacuguuguagau

SEQ ID NO: 53 - RNA, 22 nt, Homo sapiens
cuguacagccuccuagcuuucc

SEQ ID NO: 54 - RNA, 22 nt, Homo sapiens
uggaauguaaagaaguauguau

SEQ ID NO: 55 - RNA, 22 nt, Homo sapiens
caagcuuguaucuauagguaug

SEQ ID NO: 56 - RNA, 23 nt, Homo sapiens
ggcuucuuuacagugcugccuug

SEQ ID NO: 57 - RNA, 22 nt, Homo sapiens
acggauguuugagcaugugcua

SEQ ID NO: 58 - RNA, 22 nt, Homo sapiens
cugcaauguaagcacuucuuac

SEQ ID NO: 59 - RNA, 23 nt, Homo sapiens
aaaagugcuuacagugcagguag

SEQ ID NO: 60 - RNA, 22 nt, Homo sapiens
ccgcacuguggguacuugcugc

SEQ ID NO: 61 - RNA, 21 nt, Homo sapiens
uaaagugcugacagugcagau

SEQ ID NO: 62 - RNA, 22 nt, Homo sapiens
cagugcaauguuaaaagggcau

SEQ ID NO: 63 - RNA, 22 nt, Homo sapiens
gcucuuuucacauugugcuacu

SEQ ID NO: 64 - RNA, 22 nt, Homo sapiens
uuugguccccuucaaccagcug

SEQ ID NO: 65 - RNA, 22 nt, Homo sapiens
agcugguaaaauggaaccaaau

SEQ ID NO: 66 - RNA, 23 nt, Homo sapiens
uauggcuuuuuauuccuauguga

SEQ ID NO: 67 - RNA, 23 nt, Homo sapiens
uguaguguuuccuacuuuaugga

SEQ ID NO: 68 - RNA, 21 nt, Homo sapiens
cauaaaguagaaagcacuacu

SEQ ID NO: 69 - RNA, 22 nt, Homo sapiens
cuccuacauauuagcauuaaca

SEQ ID NO: 70 - RNA, 22 nt, Homo sapiens
caggccauauugugcugccuca

SEQ ID NO: 71 - RNA, 22 nt, Homo sapiens
uagcagcacguaaauauuggcg

SEQ ID NO: 72 - RNA, 22 nt, Homo sapiens
acugcagugaaggcacuuguag

SEQ ID NO: 73 - RNA, 22 nt, Homo sapiens
accacugaccguugacuguacc

SEQ ID NO: 74 - RNA, 22 nt, Homo sapiens
acugcauuaugagcacuuaaag

SEQ ID NO: 75 - RNA, 21 nt, Homo sapiens
caacaccagucgaugggcugu

SEQ ID NO: 76 - RNA, 22 nt, Homo sapiens
aguucuucaguggcaagcuuua

SEQ ID NO: 77 - RNA, 23 nt, Homo sapiens
agcuacauugucugcuggguuuc

SEQ ID NO: 78 - RNA, 22 nt, Homo sapiens
accuggcauacaauguagauuu

SEQ ID NO: 79 - RNA, 21 nt, Homo sapiens
aucacauugccagggauuucc

SEQ ID NO: 80 - RNA, 22 nt, Homo sapiens
ccuauucuugguuacuugcacg

SEQ ID NO: 81 - RNA, 24 nt, Homo sapiens
gcugguuucauauggugguuuaga

SEQ ID NO: 82 - RNA, 22 nt, Homo sapiens
uauacaagggcagacucucucu

SEQ ID NO: 83 - RNA, 22 nt, Homo sapiens
acuuuaacauggaggcacuugc

SEQ ID NO: 84 - RNA, 22 nt, Homo sapiens
cuuucagucggauguuugcagc

SEQ ID NO: 85 - RNA, 22 nt, Homo sapiens
ugcuaugccaacauauugccau

SEQ ID NO: 86 - RNA, 22 nt, Homo sapiens
caauuuagugugugugauauuu

SEQ ID NO: 87 - RNA, 18 nt, Homo sapiens
aaagcuggguugagaagg

SEQ ID NO: 88 - RNA, 21 nt, Homo sapiens
cacauuacacggucgaccucu

SEQ ID NO: 89 - RNA, 23 nt, Homo sapiens
cccacugccccaggugcugcugg

SEQ ID NO: 90 - RNA, 23 nt, Homo sapiens
ccuaguagguguccaguaagugu

SEQ ID NO: 91 - RNA, 23 nt, Homo sapiens
gggggggcaggaggggcucaggg

SEQ ID NO: 92 - RNA, 23 nt, Homo sapiens
gagguuuucuggguuucuguuuc

SEQ ID NO: 93 - RNA, 22 nt, Homo sapiens
ucucugggccugugucuuaggc

SEQ ID NO: 94 - RNA, 21 nt, Homo sapiens
gccccugggccuauccuagaa

SEQ ID NO: 95 - RNA, 22 nt, Homo sapiens
uuuuucauuauugcuccugacc

SEQ ID NO: 96 - RNA, 21 nt, Homo sapiens
gaacggcuucauacaggaguu

SEQ ID NO: 97 - RNA, 22 nt, Homo sapiens
uccagcaucagugauuuuguug

SEQ ID NO: 98 - RNA, 23 nt, Homo sapiens
ucccuguccuccaggagcucacg

SEQ ID NO: 99 - RNA, 22 nt, Homo sapiens
caauguuuccacagugcaucac

SEQ ID NO: 100 - RNA, 22 nt, Homo sapiens
uuauaaagcaaugagacugauu

SEQ ID NO: 101 - RNA, 23 nt, Homo sapiens
ucucacacagaaaucgcacccgu

SEQ ID NO: 102 - RNA, 22 nt, Homo sapiens
gcugacuccuaguccagggcuc

SEQ ID NO: 103 - RNA, 23 nt, Homo sapiens
ugucugcccgcaugccugccucu

SEQ ID NO: 104 - RNA, 22 nt, Homo sapiens
caaucagcaaguauacugcccu

SEQ ID NO: 105 - RNA, 21 nt, Homo sapiens
ccucuacagggaagcgcuuuc

SEQ ID NO: 106 - RNA, 22 nt, Homo sapiens
aaagugcuucccuuuggacugu

SEQ ID NO: 107 - RNA, 22 nt, Homo sapiens
cuacaaagggaagcacuuucuc

SEQ ID NO: 108 - RNA, 22 nt, Homo sapiens
gaaggcgcuucccuuuagagcg

SEQ ID NO: 109 - RNA, 22 nt, Homo sapiens
caaaguuuaagauccuugaagu

SEQ ID NO: 110 - RNA, 21 nt, Homo sapiens
ugaguuggccaucugagugag

SEQ ID NO: 111 - RNA, 20 nt, Homo sapiens
guccgcucggcgguggccca

SEQ ID NO: 112 - RNA, 24 nt, Homo sapiens
cugaagugauguguaacugaucag

SEQ ID NO: 113 - RNA, 23 nt, Homo sapiens
acuuacagacaagagccuugcuc

SEQ ID NO: 114 - RNA, 22 nt, Homo sapiens
uggucuaggauuguuggaggag

SEQ ID NO: 115 - RNA, 23 nt, Homo sapiens
gacacgggcgacagcugcggccc

SEQ ID NO: 116 - RNA, 22 nt, Homo sapiens
cacacacugcaauuacuuuugc

SEQ ID NO: 117 - RNA, 19 nt, Homo sapiens
aggcugcggaauucaggac

SEQ ID NO: 118 - RNA, 22 nt, Homo sapiens
agaaggcacuaugagauuuaga

SEQ ID NO: 119 - RNA, 22 nt, Homo sapiens
augcugacauauuuacuagagg

SEQ ID NO: 120 - RNA, 22 nt, Homo sapiens
guucucccaacguaagcccagc

SEQ ID NO: 121 - RNA, 21 nt, Homo sapiens
uggguuuacguugggagaacu

SEQ ID NO: 122 - RNA, 22 nt, Homo sapiens
aguauucuguaccagggaaggu

SEQ ID NO: 123 - RNA, 21 nt, Homo sapiens
agaccuggcccagaccucagc

SEQ ID NO: 124 - RNA, 19 nt, Homo sapiens
gugucugcuuccuguggga

SEQ ID NO: 125 - RNA, 23 nt, Homo sapiens
cuaauaguaucuaccacaauaaa

SEQ ID NO: 126 - RNA, 22 nt, Homo sapiens
aaccagcaccccaacuuuggac

SEQ ID NO: 127 - RNA, 23 nt, Homo sapiens
acuugggcacugaaacaaugucc

SEQ ID NO: 128 - RNA, 23 nt, Homo sapiens
ugugcuugcucgucccgcccgca

SEQ ID NO: 129 - RNA, 24 nt, Homo sapiens
acugggggcuuucgggcucugcgu

SEQ ID NO: 130 - RNA, 25 nt, Homo sapiens
agggaucgcgggcggguggcggccu

SEQ ID NO: 131 - RNA, 23 nt, Homo sapiens
aucgcugcgguugcgagcgcugu

SEQ ID NO: 132 - RNA, 21 nt, Homo sapiens
augauccaggaaccugccucu

SEQ ID NO: 133 - RNA, 24 nt, Homo sapiens
aaagacauaggauagagucaccuc

SEQ ID NO: 134 - RNA, 22 nt, Homo sapiens
auaaagcuagauaaccgaaagu

SEQ ID NO: 135 - RNA, 23 nt, Homo sapiens
agguugggaucgguugcaaugcu

SEQ ID NO: 136 - RNA, 22 nt, Homo sapiens
ggguggggauuuguugcauuac

SEQ ID NO: 137 - RNA, 23 nt, Homo sapiens
caaagugcuguucgugcagguag

SEQ ID NO: 138 - RNA, 22 nt, Homo sapiens
uucaacggguauuuauugagca

SEQ ID NO: 139 - RNA, 23 nt, Homo sapiens
uuuggcacuagcacauuuuugcu

SEQ ID NO: 140 - RNA, 22 nt, Homo sapiens
ugagguaguaaguuguauuguu

SEQ ID NO: 141 - RNA, 22 nt, Homo sapiens
caagcucgcuucuaugggucug

SEQ ID NO: 142 - RNA, 23 nt, Astatotilapia burtoni
uaaagugcuuacagugcagguag

SEQ ID NO: 143 - RNA, 21 nt, Astatotilapia burtoni
guaguguuuccuacuuuaugg

SEQ ID NO: 144 - RNA, 21 nt, Anolis carolinensis
uguaguguuuccuacuuuaug

SEQ ID NO: 145 - RNA, 23 nt, Artibeus jamaicensis
uguaguguuuccuacuuuaugga

SEQ ID NO: 146 - RNA, 23 nt, Alligator mississippiensis
uguaguguuuccuacuuuaugga

SEQ ID NO: 147 - RNA, 22 nt, Bos taurus
aaaagugcuuacagugcaggua

SEQ ID NO: 148 - RNA, 22 nt, Bos taurus
aguguuuccuacuuuauggaug

SEQ ID NO: 149 - RNA, 21 nt, Cyprinus carpio
guaguguuuccuacuuuaugg

SEQ ID NO: 150 - RNA, 21 nt, Cricetulus griseus
guaguguuuccuacuuuaugg

SEQ ID NO: 151 - RNA, 22 nt, Callithrix jacchus
guaguguuuccuacuuuaugga

SEQ ID NO: 152 - RNA, 23 nt, Columba livia
aaaagugcuuacagugcagguag

SEQ ID NO: 153 - RNA, 21 nt, Columba livia
uaguguuuccuacuuuaugga

SEQ ID NO: 154 - RNA, 23 nt, Chrysemys picta
aaaagugcuuacagugcagguag

SEQ ID NO: 155 - RNA, 22 nt, Chrysemys picta
uguaguguuuccuacuuuaugg

SEQ ID NO: 156 - RNA, 23 nt, Cavia porcellus
aaaagugcuuacagugcagguag

SEQ ID NO: 157 - RNA, 21 nt, Cavia porcellus
uaguguuuccuacuuuaugga

SEQ ID NO: 158 - RNA, 21 nt, Dasypus novemcinctus
uaguguuuccuacuuuaugga

SEQ ID NO: 159 - RNA, 23 nt, Danio rerio
uguaguguuuccuacuuuaugga

SEQ ID NO: 160 - RNA, 23 nt, Equus caballus
uguaguguuuccuacuuuaugga

SEQ ID NO: 161 - RNA, 23 nt, Eptesicus fuscus
uguaguguuuccuacuuuaugga

SEQ ID NO: 162 - RNA, 22 nt, Gallus gallus
aaaagugcuuacagugcaggua

SEQ ID NO: 163 - RNA, 22 nt, Gallus gallus
uguaguguuuccuacuuuaugg

SEQ ID NO: 164 - RNA, 22 nt, Gadus morhua
uguaguguuuccuacuuuaugg

SEQ ID NO: 165 - RNA, 23 nt, Homo sapiens
aaaagugcuuacagugcagguag

SEQ ID NO: 166 - RNA, 23 nt, Human alphaherpesvirus 1
uacaccccccugccuuccacccu

SEQ ID NO: 167 - RNA, 22 nt, Ictalurus punctatus
guaguguuuccuacuuuaugga

SEQ ID NO: 168 - RNA, 23 nt, Monodelphis domestica
uguaguguuuccuacuuuaugga

SEQ ID NO: 169 - RNA, 23 nt, Macaca mulatta
uguaguguuuccuacuuuaugga

SEQ ID NO: 170 - RNA, 23 nt, Mus musculus
caaagugcuaacagugcagguag

SEQ ID NO: 171 - RNA, 23 nt, Mus musculus
uguaguguuuccuacuuuaugga

SEQ ID NO: 172 - RNA, 22 nt, Metriaclima zebra
uggaauguaaagaaguauguau

SEQ ID NO: 173 - RNA, 23 nt, Metriaclima zebra
uaaagugcuuacagugcagguag

SEQ ID NO: 174 - RNA, 21 nt, Ornithorhynchus anatinus
aaaagugcuuacagugcaggu

SEQ ID NO: 175 - RNA, 22 nt, Ornithorhynchus anatinus
uguaguguuuccuacuuuaugg

SEQ ID NO: 176 - RNA, 22 nt, Ornithorhynchus anatinus
uguaguguuuccuacuuuaugg

SEQ ID NO: 177 - RNA, 21 nt, Ovis aries
aaaagugcuuacagugcaggu

SEQ ID NO: 178 - RNA, 23 nt, Oryctolagus cuniculus
aaaagugcuuacagugcagguag

SEQ ID NO: 179 - RNA, 21 nt, Oryctolagus cuniculus
uaguguuuccuacuuuaugga

SEQ ID NO: 180 - RNA, 23 nt, Ophiophagus hannah
uguaguguuuccuacuuuaugga

SEQ ID NO: 181 - RNA, 23 nt, Oreochromis niloticus
uaaagugcuuacagugcagguag

SEQ ID NO: 182 - RNA, 23 nt, Pteropus alecto
aaaagugcuuacagugcagguag

SEQ ID NO: 183 - RNA, 22 nt, Pteropus alecto
aguguuuccuacuuuauggaug

SEQ ID NO: 184 - RNA, 21 nt, Python bivittatus
uaguguuuccuacuuuaugga

SEQ ID NO: 185 - RNA, 23 nt, Haplochromis nyererei
uaaagugcuuacagugcagguag

SEQ ID NO: 186 - RNA, 23 nt, Pongo pygmaeus
uguaguguuuccuacuuuaugga

SEQ ID NO: 187 - RNA, 23 nt, Pan troglodytes
uguaguguuuccuacuuuaugga

SEQ ID NO: 188 - RNA, 23 nt, Rattus norvegicus
uguaguguuuccuacuuuaugga

SEQ ID NO: 189 - RNA, 22 nt, Salmo salar
uaaagugcuuacagugcaggua

SEQ ID NO: 190 - RNA, 22 nt, Salmo salar
aaaagugcuuacagugcaggua

SEQ ID NO: 191 - RNA, 22 nt, Salmo salar
aguguuuccuacuuuauggaug

SEQ ID NO: 192 - RNA, 24 nt, Salmo salar
uguaguguuuccuacuuuauggau

SEQ ID NO: 193 - RNA, 22 nt, Sus scrofa
uguaguguuuccuacuuuaugg

SEQ ID NO: 194 - RNA, 22 nt, Tupaia chinensis
guaguguuuccuacuuuaugga

SEQ ID NO: 195 - RNA, 23 nt, Taeniopygia guttata
aaaagugcuuacagugcagguag

SEQ ID NO: 196 - RNA, 22 nt, Taeniopygia guttata
guaguguuuccuacuuuaugga

SEQ ID NO: 197 - RNA, 23 nt, Xenopus laevis
uguaguguuuccuacuuuaugga

SEQ ID NO: 198 - RNA, 23 nt, Xenopus tropicalis
uguaguguuuccuacuuuaugga

SEQ ID NO: 199 - DNA, 22 bp, Homo sapiens
ggaaagctaggaggctgtacag

SEQ ID NO: 200 - DNA, 22 bp, Homo sapiens
atacatacttctttacattcca

SEQ ID NO: 201 - DNA, 22 bp, Homo sapiens
catacctatagatacaagcttg

SEQ ID NO: 202 - DNA, 23 bp, Homo sapiens
caaggcagcactgtaaagaagcc

SEQ ID NO: 203 - DNA, 22 bp, Homo sapiens
tagcacatgctcaaacatccgt

SEQ ID NO: 204 - DNA, 22 bp, Homo sapiens
gtaagaagtgcttacattgcag

SEQ ID NO: 205 - DNA, 23 bp, Homo sapiens
ctacctgcactgtaagcactttt

SEQ ID NO: 206 - DNA, 22 bp, Homo sapiens
gcagcaagtacccacagtgcgg

SEQ ID NO: 207 - DNA, 21 bp, Homo sapiens
atctgcactgtcagcacttta

SEQ ID NO: 208 - DNA, 22 bp, Homo sapiens
atgcccttttaacattgcactg

SEQ ID NO: 209 - DNA, 22 bp, Homo sapiens
agtagcacaatgtgaaaagagc

SEQ ID NO: 210 - DNA, 22 bp, Homo sapiens
cagctggttgaaggggaccaaa

SEQ ID NO: 211 - DNA, 22 bp, Homo sapiens
atttggttccattttaccagct

SEQ ID NO: 212 - DNA, 23 bp, Homo sapiens
tcacataggaataaaaagccata

SEQ ID NO: 213 - DNA, 23 bp, Homo sapiens
tccataaagtaggaaacactaca

SEQ ID NO: 214 - DNA, 21 bp, Homo sapiens
agtagtgctttctactttatg

SEQ ID NO: 215 - DNA, 22 bp, Homo sapiens
tgttaatgctaatatgtaggag

SEQ ID NO: 216 - DNA, 22 bp, Homo sapiens
tgaggcagcacaatatggcctg

SEQ ID NO: 217 - DNA, 22 bp, Homo sapiens
cgccaatatttacgtgctgcta

SEQ ID NO: 218 - DNA, 22 bp, Homo sapiens
ctacaagtgccttcactgcagt

SEQ ID NO: 219 - DNA, 22 bp, Homo sapiens
ggtacagtcaacggtcagtggt

SEQ ID NO: 220 - DNA, 22 bp, Homo sapiens
ctttaagtgctcataatgcagt

SEQ ID NO: 221 - DNA, 21 bp, Homo sapiens
acagcccatcgactggtgttg

SEQ ID NO: 222 - DNA, 22 bp, Homo sapiens
taaagcttgccactgaagaact

SEQ ID NO: 223 - DNA, 23 bp, Homo sapiens
gaaacccagcagacaatgtagct

SEQ ID NO: 224 - DNA, 22 bp, Homo sapiens
aaatctacattgtatgccaggt

SEQ ID NO: 225 - DNA, 21 bp, Homo sapiens
ggaaatccctggcaatgtgat

SEQ ID NO: 226 - DNA, 22 bp, Homo sapiens
cgtgcaagtaaccaagaatagg

SEQ ID NO: 227 - DNA, 24 bp, Homo sapiens
tctaaaccaccatatgaaaccagc

SEQ ID NO: 228 - DNA, 22 bp, Homo sapiens
agagagagtctgcccttgtata

SEQ ID NO: 229 - DNA, 22 bp, Homo sapiens
gcaagtgcctccatgttaaagt

SEQ ID NO: 230 - DNA, 22 bp, Homo sapiens
gctgcaaacatccgactgaaag

SEQ ID NO: 231 - DNA, 22 bp, Homo sapiens
atggcaatatgttggcatagca

SEQ ID NO: 232 - DNA, 22 bp, Homo sapiens
aaatatcacacacactaaattg

SEQ ID NO: 233 - DNA, 18 bp, Homo sapiens
ccttctcaacccagcttt

SEQ ID NO: 234 - DNA, 21 bp, Homo sapiens
agaggtcgaccgtgtaatgtg

SEQ ID NO: 235 - DNA, 23 bp, Homo sapiens
ccagcagcacctggggcagtggg

SEQ ID NO: 236 - DNA, 23 bp, Homo sapiens
acacttactggacacctactagg

SEQ ID NO: 237 - DNA, 23 bp, Homo sapiens
ccctgagcccctcctgccccccc

SEQ ID NO: 238 - DNA, 23 bp, Homo sapiens
gaaacagaaacccagaaaacctc

SEQ ID NO: 239 - DNA, 22 bp, Homo sapiens
gcctaagacacaggcccagaga

SEQ ID NO: 240 - DNA, 21 bp, Homo sapiens
ttctaggataggcccaggggc

SEQ ID NO: 241 - DNA, 22 bp, Homo sapiens
ggtcaggagcaataatgaaaaa

SEQ ID NO: 242 - DNA, 21 bp, Homo sapiens
aactcctgtatgaagccgttc

SEQ ID NO: 243 - DNA, 22 bp, Homo sapiens
caacaaaatcactgatgctgga

SEQ ID NO: 244 - DNA, 23 bp, Homo sapiens
cgtgagctcctggaggacaggga

SEQ ID NO: 245 - DNA, 22 bp, Homo sapiens
gtgatgcactgtggaaacattg

SEQ ID NO: 246 - DNA, 22 bp, Homo sapiens
aatcagtctcattgctttataa

SEQ ID NO: 247 - DNA, 23 bp, Homo sapiens
acgggtgcgatttctgtgtgaga

SEQ ID NO: 248 - DNA, 22 bp, Homo sapiens
gagccctggactaggagtcagc

SEQ ID NO: 249 - DNA, 23 bp, Homo sapiens
agaggcaggcatgcgggcagaca

SEQ ID NO: 250 - DNA, 22 bp, Homo sapiens
agggcagtatacttgctgattg

SEQ ID NO: 251 - DNA, 21 bp, Homo sapiens
gaaagcgcttccctgtagagg

SEQ ID NO: 252 - DNA, 22 bp, Homo sapiens
acagtccaaagggaagcacttt

SEQ ID NO: 253 - DNA, 22 bp, Homo sapiens
gagaaagtgcttccctttgtag

SEQ ID NO: 254 - DNA, 22 bp, Homo sapiens
cgctctaaagggaagcgccttc

SEQ ID NO: 255 - DNA, 22 bp, Homo sapiens
acttcaaggatcttaaactttg

SEQ ID NO: 256 - DNA, 21 bp, Homo sapiens
ctcactcagatggccaactca

SEQ ID NO: 257 - DNA, 20 bp, Homo sapiens
tgggccaccgccgagcggac

SEQ ID NO: 258 - DNA, 24 bp, Homo sapiens
ctgatcagttacacatcacttcag

SEQ ID NO: 259 - DNA, 23 bp, Homo sapiens
gagcaaggctcttgtctgtaagt

SEQ ID NO: 260 - DNA, 22 bp, Homo sapiens
ctcctccaacaatcctagacca

SEQ ID NO: 261 - DNA, 23 bp, Homo sapiens
gggccgcagctgtcgcccgtgtc

SEQ ID NO: 262 - DNA, 22 bp, Homo sapiens
gcaaaagtaattgcagtgtgtg

SEQ ID NO: 263 - DNA, 19 bp, Homo sapiens
gtcctgaattccgcagcct

SEQ ID NO: 264 - DNA, 22 bp, Homo sapiens
tctaaatctcatagtgccttct

SEQ ID NO: 265 - DNA, 22 bp, Homo sapiens
cctctagtaaatatgtcagcat

SEQ ID NO: 266 - DNA, 22 bp, Homo sapiens
gctgggcttacgttgggagaac

SEQ ID NO: 267 - DNA, 21 bp, Homo sapiens
agttctcccaacgtaaaccca

SEQ ID NO: 268 - DNA, 22 bp, Homo sapiens
accttccctggtacagaatact

SEQ ID NO: 269 - DNA, 21 bp, Homo sapiens
gctgaggtctgggccaggtct

SEQ ID NO: 270 - DNA, 19 bp, Homo sapiens
tcccacaggaagcagacac

SEQ ID NO: 271 - DNA, 23 bp, Homo sapiens
tttattgtggtagatactattag

SEQ ID NO: 272 - DNA, 22 bp, Homo sapiens
gtccaaagttggggtgctggtt

SEQ ID NO: 273 - DNA, 23 bp, Homo sapiens
ggacattgtttcagtgcccaagt

SEQ ID NO: 274 - DNA, 23 bp, Homo sapiens
tgcgggcgggacgagcaagcaca

SEQ ID NO: 275 - DNA, 24 bp, Homo sapiens
acgcagagcccgaaagcccccagt

SEQ ID NO: 276 - DNA, 25 bp, Homo sapiens
aggccgccacccgcccgcgatccct

SEQ ID NO: 277 - DNA, 23 bp, Homo sapiens
acagcgctcgcaaccgcagcgat

SEQ ID NO: 278 - DNA, 21 bp, Homo sapiens
agaggcaggttcctggatcat

SEQ ID NO: 279 - DNA, 24 bp, Homo sapiens
gaggtgactctatcctatgtcttt

SEQ ID NO: 280 - DNA, 22 bp, Homo sapiens
actttcggttatctagctttat

SEQ ID NO: 281 - DNA, 23 bp, Homo sapiens
agcattgcaaccgatcccaacct

SEQ ID NO: 282 - DNA, 22 bp, Homo sapiens
gtaatgcaacaaatccccaccc

SEQ ID NO: 283 - DNA, 23 bp, Homo sapiens
ctacctgcacgaacagcactttg

SEQ ID NO: 284 - DNA, 22 bp, Homo sapiens
tgctcaataaatacccgttgaa

SEQ ID NO: 285 - DNA, 23 bp, Homo sapiens
agcaaaaatgtgctagtgccaaa

SEQ ID NO: 286 - DNA, 22 bp, Homo sapiens
aacaatacaacttactacctca

SEQ ID NO: 287 - DNA, 22 bp, Homo sapiens
cagacccatagaagcgagcttg

SEQ ID NO: 288 - DNA, 23 bp, Astatotilapia burtoni
ctacctgcactgtaagcacttta

SEQ ID NO: 289 - DNA, 21 bp, Astatotilapia burtoni
ccataaagtaggaaacactac

SEQ ID NO: 290 - DNA, 21 bp, Anolis carolinensis
cataaagtaggaaacactaca

SEQ ID NO: 291 - DNA, 23 bp, Artibeus jamaicensis
tccataaagtaggaaacactaca

SEQ ID NO: 292 - DNA, 23 bp, Alligator mississippiensis
tccataaagtaggaaacactaca

SEQ ID NO: 293 - DNA, 22 bp, Bos taurus
tacctgcactgtaagcactttt

SEQ ID NO: 294 - DNA, 22 bp, Bos taurus
catccataaagtaggaaacact

SEQ ID NO: 295 - DNA, 21 bp, Cyprinus carpio
ccataaagtaggaaacactac

SEQ ID NO: 296 - DNA, 21 bp, Cricetulus griseus
ccataaagtaggaaacactac

SEQ ID NO: 297 - DNA, 22 bp, Callithrix jacchus
tccataaagtaggaaacactac

SEQ ID NO: 298 - DNA, 23 bp, Columba livia
ctacctgcactgtaagcactttt

SEQ ID NO: 299 - DNA, 21 bp, Columba livia
tccataaagtaggaaacacta

SEQ ID NO: 300 - DNA, 23 bp, Chrysemys picta
ctacctgcactgtaagcactttt

SEQ ID NO: 301 - DNA, 22 bp, Chrysemys picta
ccataaagtaggaaacactaca

SEQ ID NO: 302 - DNA, 23 bp, Cavia porcellus
ctacctgcactgtaagcactttt

SEQ ID NO: 303 - DNA, 21 bp, Cavia porcellus
tccataaagtaggaaacacta

SEQ ID NO: 304 - DNA, 21 bp, Dasypus novemcinctus
tccataaagtaggaaacacta

SEQ ID NO: 305 - DNA, 23 bp, Danio rerio
tccataaagtaggaaacactaca

SEQ ID NO: 306 - DNA, 23 bp, Equus caballus
tccataaagtaggaaacactaca

SEQ ID NO: 307 - DNA, 23 bp, Eptesicus fuscus
tccataaagtaggaaacactaca

SEQ ID NO: 308 - DNA, 22 bp, Gallus gallus
tacctgcactgtaagcactttt

SEQ ID NO: 309 - DNA, 22 bp, Gallus gallus
ccataaagtaggaaacactaca

SEQ ID NO: 310 - DNA, 22 bp, Gadus morhua
ccataaagtaggaaacactaca

SEQ ID NO: 311 - DNA, 23 bp, Homo sapiens
ctacctgcactgtaagcactttt

SEQ ID NO: 312 - DNA, 23 bp, Human alphaherpesvirus 1
agggtggaaggcaggggggtgta

SEQ ID NO: 313 - DNA, 22 bp, Ictalurus punctatus
tccataaagtaggaaacactac

SEQ ID NO: 314 - DNA, 23 bp, Monodelphis domestica
tccataaagtaggaaacactaca

SEQ ID NO: 315 - DNA, 23 bp, Macaca mulatta
tccataaagtaggaaacactaca

SEQ ID NO: 316 - DNA, 23 bp, Mus musculus
ctacctgcactgttagcactttg

SEQ ID NO: 317 - DNA, 23 bp, Mus musculus
tccataaagtaggaaacactaca

SEQ ID NO: 318 - DNA, 22 bp, Metriaclima zebra
atacatacttctttacattcca

SEQ ID NO: 319 - DNA, 23 bp, Metriaclima zebra
ctacctgcactgtaagcacttta

SEQ ID NO: 320 - DNA, 21 bp, Ornithorhynchus anatinus
acctgcactgtaagcactttt

SEQ ID NO: 321 - DNA, 22 bp, Ornithorhynchus anatinus
ccataaagtaggaaacactaca

SEQ ID NO: 322 - DNA, 22 bp, Ornithorhynchus anatinus
ccataaagtaggaaacactaca

SEQ ID NO: 323 - DNA, 21 bp, Ovis aries
acctgcactgtaagcactttt

SEQ ID NO: 324 - DNA, 23 bp, Oryctolagus cuniculus
ctacctgcactgtaagcactttt

SEQ ID NO: 325 - DNA, 21 bp, Oryctolagus cuniculus
tccataaagtaggaaacacta

SEQ ID NO: 326 - DNA, 23 bp, Ophiophagus hannah
tccataaagtaggaaacactaca

SEQ ID NO: 327 - DNA, 23 bp, Oreochromis niloticus
ctacctgcactgtaagcacttta

SEQ ID NO: 328 - DNA, 23 bp, Pteropus alecto
ctacctgcactgtaagcactttt

SEQ ID NO: 329 - DNA, 22 bp, Pteropus alecto
catccataaagtaggaaacact

SEQ ID NO: 330 - DNA, 21 bp, Python bivittatus
tccataaagtaggaaacacta

SEQ ID NO: 331 - DNA, 23 bp, Haplochromis nyererei
ctacctgcactgtaagcacttta

SEQ ID NO: 332 - DNA, 23 bp, Pongo pygmaeus
tccataaagtaggaaacactaca

SEQ ID NO: 333 - DNA, 23 bp, Pan troglodytes
tccataaagtaggaaacactaca

SEQ ID NO: 334 - DNA, 23 bp, Rattus norvegicus
tccataaagtaggaaacactaca

SEQ ID NO: 335 - DNA, 22 bp, Salmo salar
tacctgcactgtaagcacttta

SEQ ID NO: 336 - DNA, 22 bp, Salmo salar
tacctgcactgtaagcactttt

SEQ ID NO: 337 - DNA, 22 bp, Salmo salar
catccataaagtaggaaacact

SEQ ID NO: 338 - DNA, 24 bp, Salmo salar
atccataaagtaggaaacactaca

SEQ ID NO: 339 - DNA, 22 bp, Sus scrofa
ccataaagtaggaaacactaca

SEQ ID NO: 340 - DNA, 22 bp, Tupaia chinensis
tccataaagtaggaaacactac

SEQ ID NO: 341 - DNA, 23 bp, Taeniopygia guttata
ctacctgcactgtaagcactttt

SEQ ID NO: 342 - DNA, 22 bp, Taeniopygia guttata
tccataaagtaggaaacactac

SEQ ID NO: 343 - DNA, 23 bp, Xenopus laevis
tccataaagtaggaaacactaca

SEQ ID NO: 344 - DNA, 23 bp, Xenopus tropicalis
tccataaagtaggaaacactaca

SEQ ID NO: 345 - DNA, 20 bp, Artificial Sequence
  Description of Artificial Sequence Synthetic probe
gctggatagcctccaggcca

SEQ ID NO: 346 - DNA, 23 bp, Artificial Sequence
  Description of Artificial Sequence Synthetic probe
gccatgctaatcttctctgtatc

(19) United States
(12) Patent Application Publication
(10) Pub. No.: US 2023/0088902 A1
(43) Pub. Date: Mar. 23, 2023

(54) CELL SPECIFIC, SELF-INACTIVATING GENOMIC EDITING USING CRISPR-CAS SYSTEMS HAVING RNASE AND DNASE ACTIVITY

(71) Applicant: Icahn School of Medicine at Mount Sinai, New York, NY (US)

(72) Inventors: Benjamin R. Tenoever, New York, NY (US); Rasmus Moeller, New York, NY (US)

(21) Appl. No.: 17/759,564

(22) PCT Filed: Jan. 27, 2021
(86) PCT No.: PCT/US21/15216

§ 371 Date: Jul. 27, 2022

Related U.S. Application Data
(60) Provisional application No. 62/966,585, filed on Jan. 28, 2020.

(51) Int. Cl. C12N 9/22; C12N 15/11; C12N 15/90

(52) U.S. Cl. CPC C12N 9/22; C12N 15/11; C12N 15/907; C12N 2310/20

(57) ABSTRACT

This disclosure provides a CRISPR-Cas system with both RNase and Dnase activity for genetic editing and methods of use thereof. The disclosed CRISPR-Cas system can function in a cell-specific manner, which enables in vivo editing while mitigating the risk of off-target effects.

### FIELD OF THE INVENTION

This disclosure relates generally to a Clustered Regularly Interspersed Short Palindromic Repeat (CRISPR)-CRISPR associated (Cas) (CRISPR-Cas) system for genetic editing and more specifically to a CRISPR-Cas system with both RNAse and DNAse activity that can be engineered to both self-inactivate and function with cell specificity and methods of use thereof.

### BACKGROUND OF THE INVENTION

The CRISPR-Cas systems of archaea and many bacteria are sequence-specific adaptive defense systems that have evolved to cleave foreign nucleic acid (Marraffini, L. A. & Sontheimer, E. J. Nat Rev Genet 11, 181-190 (2010)). This defense system is dependent on acquisition and integration of foreign DNA spacers in a process generally referred to as adaptation (Makarova, K. S. et al. Nat Rev Microbiol 13, 722-736 (2015)). Once integrated, expression of the so-called protospacers generates a precursor CRISPR RNA (pre-crRNA), which is further processed and matured to produce crRNA. Finally, crRNA is bound by a Cas nuclease to elicit interference on incoming DNA as defined by complementarity of its guide RNA. Moreover, as the protospacer DNA is inherited, adaptation of a single prokaryotic cell can result in Lamarckian evolution for its offspring (van der Oost, et al. Trends Biochem Sci 34, 401-407 (2009)).

While most of the Cas-nucleases only possess RNA-guided DNase activity, Cas12a and a subset of other also have RNase function (Fonfara, I., et al. Nature 532, 517-521 (2016)). The RNase function is responsible for processing the pre-crRNA by cleaving direct repeat sequences that flank this 20 nucleotide sequence (Fonfara, I., et al. Nature 532, 517-521 (2016)). The crRNA that is generated as a result of these processing events is sufficient for instilling specificity onto the DNase activity of Cas12a. Similar to Cas9, Cas12a has also been repurposed as a eukaryotic gene editor (Cho, S. W., et al. Nat Biotechnol 31, 230-232 (2013); Cong, L. et al. Science 339, 819-823 (2013)). However, as Cas12a biology is still in its infancy, its optimization lags behind that of Cas9. Despite this, the ability of Cas12a to process its own crRNA enables one to use it to generate the crRNA from diverse types of RNA so long as it is flanked by direct repeats (Zetsche, B. et al. Nat Biotechnol 35, 31-34 (2017)). This activity not only allows one to generate multiple crRNAs for any number of targets, but it has also enabled the generation of mRNAs that both code for Cas12a and the desired guides on a single transcript (Campa, C. C., et al. Nat Methods 16, 887-893 (2019)). This is in contrast to the Cas9 system, which demands a separate DNA-dependent RNA polymerase for production of Cas9 and the single guide RNA (Cong, L. et al. Science 339, 819-823 (2013); Jinek, M. et al. Science 337, 816-821 (2012)).

Despite the immense potential of both the CRISPR-Cas systems, one significant impediment is delivery of these large proteins alongside the desired crRNA(s). This challenge is formidable, especially when one wishes to efficiently edit a large number of cells to repair a genetic defect in vivo. This problem is further confounded by the fact that maintaining Cas expression for longer periods of time can result in the generation of off-target effects, chromosomal translocations, and/or removal of the Cas-expressing cells (Koo, T., et al. Mol Cells 38, 475-481 (2015)).

Given the above challenges, there is a pressing need for optimal genetic editors that can be delivered with the efficiency of a virus in a manner that is free of genomic integration and function only in a desired cell type for the time required to achieve editing.

### SUMMARY OF THE INVENTION

This disclosure addresses the need mentioned above in a number of aspects. In one aspect, this disclosure provides a system for gene editing. The system comprises (i) a Cas nucleotide sequence encoding a CRISPR-Cas protein with both RNAse and DNase activity; and (ii) a targeting sequence comprising in 5′ to 3′ direction (a) a direct repeat sequence, (b) a guide nucleotide sequence encoding or comprising a crRNA sequence capable of hybridizing with a target sequence and forming a complex with the CRISPR-Cas protein, and (c) at least one microRNA target site capable of hybridizing with a microRNA that mediates cleavage of the microRNA-target site by a microRNA-associated protein. In some embodiments, the system is a nucleic acid, such as an RNA. In some embodiments, the guide nucleotide sequence further comprises an AU-rich element, a degradation tag, or a combination thereof, located downstream from the microRNA-target site.

In some embodiments, the Cas nucleotide sequence and the guide nucleotide sequence are located on the same vector. In some embodiments, the Cas nucleotide sequence and the guide nucleotide sequence are located on different vectors.

In some embodiments, the microRNA-target site is selected from the group consisting of SEQ ID NOs: 199-344. In some embodiments, the microRNA target site can bind to a cognate microRNA with minimum free energy (MFE) of less than −35 kcal/mol.

In some embodiments, when the crRNA sequence forms a complex with the CRISPR-Cas protein and hybridizes to the target sequence, the CRISPR-Cas protein induces distal cleavage of the target sequence.

In some embodiments, the CRISPR-Cas protein is a Cas12a protein. In some embodiments, the Cas12a protein is derived from a bacterial species selected from the group consisting of Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. In some embodiments, the Cas12a protein is PaCpf1p, LbCpf1, or AsCpf1. In some embodiments, the Cas12a protein has at least 75% sequence identity with SEQ ID NOs: 1-19. In some embodiments, the Cas12a protein comprises one or more nuclear localization signals (NLSs).

In another aspect, this disclosure provides a host cell or cell line or progeny thereof comprising the system described above. In some embodiments, the host cell or cell line or progeny thereof comprises a stem cell or stem cell line. Also provided is a composition comprising the system described above.

In yet another aspect, this disclosure further provides a method of modifying a target sequence of interest comprising delivering the system or the composition, as described above, to the target sequence or a cell containing the target sequence. In some embodiments, following formation of a complex between the crRNA sequence and the CRISPR-Cas protein and hybridization of the crRNA sequence to one or more nucleic acid of the target sequence, the CRISPR-Cas protein induces a modification of the target sequence.

In some embodiments, the cell is a eukaryotic cell, such as a plant, animal, or human cell. In some embodiments, the cell is a human stem cell.

In some embodiments, the target sequence is located at genomic loci of interest. In some embodiments, the target sequence comprises DNA. In some embodiments, the DNA is relaxed or supercoiled. In some embodiments, the target sequence is located at the 3′ end of a Protospacer Adjacent Motif (PAM). In some embodiments, the PAM comprises a 5′ T-rich motif. In some embodiments, the PAM sequence is TTN, where N is A/C/G or T.

In some embodiments, the target sequence is associated with a disease, such as a disease caused by a genetic defect in the target sequence. In some embodiments, the disease is cancer.

In some embodiments, the system or the isolated nucleic acid is delivered via particles, vesicles, or one or more viral vectors. In some embodiments, the one or more viral vectors comprise an adenovirus-based vector, a lentivirus-based vector, or an adeno-associated virus-based vector, or an RNA virus-based vector. In some embodiments, the modification of the target sequence is a strand break. In some embodiments, the target sequence is modified by the integration of a DNA insert into the staggered DNA double-stranded break.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows a schematic of a self-inactivating Cas12a delivery vector. The construct encodes for: a CMV promoter; an EGFP and Cas12a fusion protein separated by a P2A peptide; a crRNA flanked by two direct repeat sequences (DRs); and an SV40 polyadenylation site (pA).

FIG. 2 shows the validation of Cas12a-mediated vector cleavage. Constructs as described in FIG. 1 or with different variations in direct repeat, e.g., substitution of nucleotide 18 from the 3′ end of the direct repeat from adenosine to guanine (A18G); inversion of nucleotides 16-19 in the direct repeat from AAUU to UUAA; and direct repeats replaced by scrambled sequence (scrbl). The last two constructs have two target sites to either a control-miRNA (miR-142-3p, ctrl-T) or miR-106a (miR-106T). Fluorescence images are representative of GFP expression 48 hours after transfection with the constructs indicated.

FIG. 3 shows Western blot results of whole-cell extracts from fibroblasts transfected with the Cas12a-construct or Cas12a followed by crRNA flanked by the direct repeat variations indicated, or Cas12a followed by a crRNA flanked by direct repeat upstream and a miRNA target site indicated and an ARE downstream (constructs as in FIG. 2 with “dDR” (dead direct repeat) referring to the “scrbl” construct in FIG. 2). Ø: untransfected control. Blot probed with antibodies specific to HA-Cas12a, GFP, or actin.

FIG. 4 shows Northern blot results of total RNA from fibroblasts transfected with the Cas12a-construct or Cas12a followed by a crRNA flanked by the direct repeat variations indicated, or Cas12a followed by a crRNA flanked by a direct repeat upstream and a miRNA target site downstream as indicated as well as a terminal ARE (constructs as in FIG. 2). 0: untransfected control. Blot probed for B2M-specific crRNA, U6 snRNA, and miR-106a.

FIGS. 5A and 5B show cell surface expression of MHC-I and cellular expression of EGFP analyzed by flow cytometry on fibroblasts transfected with the indicated constructs. FIG. 5A. Cas12a and a B2M-specific crRNA flanked by direct repeats or repeats with nucleotides 16-19 changed from AAUU to UUAA. MHC-I positive gate is defined based on 99% of the cells transfected with the UUAA construct. Bottom panel: Overlay of the MHC-I signal from UUAA and direct repeat transfected cells. FIG. 5B. MHC Class I cell surface expression measured ten days by flow cytometry ten days post transfection. Data from cells transfected with the constructs overlaid as indicated.

FIG. 6 shows a schematic of a self-inactivating Cas12a replicon delivery vector. The construct encodes for: a Nodamuravirus RNA-dependent RNA polymerase (Noda-RdRp) fused to EGFP and Cas12a separated by P2A sites; a crRNA flanked by two direct repeats; and a 3′ replication element (3′ RE) secondary structure that facilitates Nodamuravirus replication.

FIG. 7 shows Western blot results of whole-cell extracts from fibroblasts transfected with a Nodamuravirus replicon encoding Cas12a followed by a crRNA flanked by the direct repeat variations indicated. dDR=dead direct repeat; direct repeats replaced by scrambled sequence. Ø: untransfected control. Blot probed with antibodies specific to HA-Cas12a, interferon-induced protein with tetratricopeptide repeats (IFIT1), or the housekeeping protein GAPDH.

FIG. 8 shows a schematic of a self-inactivating Cas12a delivery vector with miRNA-dependent crRNA processing. The construct encodes for: a CMV promoter; an EGFP and Cas12a fusion protein separated by a P2A site: a crRNA flanked by a 5′ direct repeat and a downstream miRNA target site (miR-T) followed by an AU-rich element (ARE); and an SV40 polyadenylation site (pA).

FIGS. 9A, 9B, 9C, and 9D show the transcriptional response to self-inactivating Cas12a vectors. FIG. 9A. Plot depicting differential gene expression of host genes in cells transfected with a plasmid-based Cas12a construct containing direct repeats in the 3′-UTR compared to cells transfected with a comparable construct without direct repeats. Each dot represents a gene plotted by its log 2 fold change between the two conditions and −log 10 of the adjusted p-value (q) determined based on triplicate samples. Horizontal line marks a q-value=0.01 and Vertical lines mark a log 2 fold change of −1 and 1. FIG. 9B. Same as FIG. 9A, but comparing replicon-based Cas12a construct containing direct repeats against a comparable construct without direct repeats. FIG. 9C. Same as FIG. 9A, but comparing plasmid-based Cas12a with direct repeats to replicon-based Cas12a with direct repeats. FIG. 9D. Stranded read numbers aligning to the replicon as number of reads per million of total reads. Error bars represent standard deviation from three replicates.

### DETAILED DESCRIPTION OF THE INVENTION

The capacity to edit genomes in a sequence-specific manner holds immense potential for countless genetic-based diseases. However, one significant impediment preventing broad therapeutic utilization is in vivo delivery. While genetic editing at a single cell level in vitro can be achieved with relatively high efficiency, the capacity to utilize these same biologic tools in a desired tissue in vivo remains challenging. In an effort to address this challenge, this disclosure describes a versatile RNA-based technology that can be adapted to diverse delivery systems and to achieve cell-specific activity by combining host microRNA biology with the CRISPR-Cas12a platform. Utilizing the RNase activity of Cas12a, this disclosure provides a self-inactivating system that utilizes cell-specific microRNAs for proper guide RNA processing and the removal of a destabilizing domain. This disclosure further demonstrates that this genetic editing circuit can function in a cell-specific manner as both an mRNA and in the context of RNA-based vectors thereby enabling in vivo editing while mitigating the risk of off-target effects.

### I. CRISPR-CAS GENE EDITING SYSTEMS WITH RNASE AND DNASE ACTIVITY

The CRISPR-Cas systems as disclosed herein encompasses a subset of CRISPR-Cas proteins (e.g., a subset of Type V CRISPR-Cas proteins) that demonstrate both RNAse and DNase activity. The Type V CRISPR-Cas systems are functionally distinct from the CRISPR-Cas9 systems. Cas12a, a member of the Type V CRISPR-Cas system, is a single RNA-guided endonuclease lacking a trans-activating crRNA (tracrRNA), and that utilizes a 5′ T-rich PAM site and cleaves DNA via a staggered DNA double-stranded break distal to the PAM site.

In one aspect, the CRISPR-Cas systems described herein comprise: (i) the open reading frame for a Cas member capable of dual RNAse and DNase activity; and (ii) a non-coding RNA sequence comprising in 5′ to 3′ direction (a) a direct repeat sequence recognizable to the cognate Cas protein, (b) a guide nucleotide sequence encoding or comprising a crRNA capable of hybridizing with a target sequence and forming a complex with the Cas protein, and (c) a second direct repeat sequence recognizable to the cognate Cas protein. In some embodiments, component (c), the second direct repeat can be replaced with at least one (e.g., 1, 2, 3, 4, 5, 6, 7, 8, 9, 10, etc.) microRNA-target site capable of hybridizing with a microRNA that mediates cleavage of the microRNA-target site by a microRNA-associated protein. In some embodiments, the system is a nucleic acid, such as an RNA. In some embodiments, the Cas nucleotide sequence and the guide nucleotide sequence are located on the same vector. In some embodiments, the Cas nucleotide sequence and the guide nucleotide sequence are located on different vectors.

a. CRISPR-Cas Proteins and Cas12a

The present invention encompasses CRISPR-Cas proteins that have both RNase and DNase activity, such as Cas12a (a type V-A Cas protein). Cas12a is a large protein with about 1,100-1,300 amino acids. Several unique features make Cas12a distinguished from Cas9, providing a substantial expansion of CRISPR-based genome-editing tools. First, Cas12a is a single crRNA-guided endonuclease, while Cas9 is guided by a dual-RNA system consisting of a crRNA and a tracrRNA. Second, Cas12a recognizes a 5′ T-rich PAM, different from the 3′ G-rich PAM utilized by Cas9. Third, after cleavage of double-stranded DNAs (dsDNAs), Cas12a generates staggered ends distal to the PAM site, whereas Cas9 introduces blunt ends within the PAM-proximal target site. Moreover, RuvC and Nuc domains of Cas12a are responsible for target DNA cleavage, whereas Cas9 uses the RuvC and HNH endonuclease domains to cleave the target DNAs.

In some embodiments, the CRISPR-Cas protein can be a mutant of a wild type Cas protein (e.g., Cas12a) or an active fragment thereof. For example, in some embodiments, Cas12a can be derived from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus. In some embodiments, Cas12a can be derived from an organism, such as S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, and C. sordellii.

In some embodiments, Cas12a can be derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In some embodiments, the Cas12a is derived from Francisella novicida, Acidaminococcus sp. (e.g., Acidaminococcus sp. BV3L6), Lachnospiraceae sp. (e.g., Lachnospiraceae bacterium MA2020), and Prevotella sp.

In some embodiments, the Cas12a protein comprises the amino acid sequence of SEQ

TABLE 1
Example Accession Codes of Cas12a Proteins

| | | NCBI Reference | SEQ |
|---|---|---|---|
| | | Sequence/GenBank | ID |
| Species | Domain | Accession No. | NO |
| Lachnospiraceae bacterium ND2006 | Bacteria | WP_051666128.1 | 1 |
| Candidatus Methanomethylophilus alvus Mx1201 | Archaea | AGI85632.1 | 2 |
| Sneatia amnii | Bacteria | WP_084710347.1 | 3 |
| Acidaminococcus sp. BV3L6 | Bacteria | WP_021736722.1 | 4 |
| Parcubacteria group bacterium GW2011 | Bacteria | KKT50231.1 | 5 |
| Candidatus Roizmanbacteria bacterium GW2011 | Bacteria | KKQ38174.1 | 6 |
| Candidatus Peregrinbacterium bacterium | Bacteria | KKP39507.1 | 7 |
| GW2011 | | | |
| Lachnospiracea bacterium MA2020 | Bacteria | WP_044919442.1 | 8 |
| Butyrivibrio sp. NC3005 | Bacteria | WP_035798880.1 | 9 |
| Butyrivibrio fibrisolvens | Bacteria | WP_027216152.1 | 10 |
| Prevotella bryantii B14 | Bacteria | SER03894.1 | 11 |
| Bacteroidetes oral taxon 274 | Bacteria | EFI15981.1 | 12 |
| Flavobacterium branchiophilum FL-15 | Bacteria | WP_014085038.1 | 13 |
| Lachnospiraceae bacterium MC2017 | Bacteria | WP_081834226.1 | 14 |
| Moraxella lacunata | Bacteria | WP_115247861.1 | 15 |
| Moraxella bovoculi AAX08_00205 | Bacteria | AKG14689.1 | 16 |
| Moraxella bovoculi AAX11_00205 | Bacteria | AKG12737.1 | 17 |
| Francisella novicida U112 | Bacteria | WP_003040289.1 | 18 |
| Thiomicrospira sp. XS5 | Bacteria | WP_068647445.1 | 19 |

In some embodiments, the CRISPR-Cas protein can be derived from a mutant Cas protein. For example, the amino acid sequence of the Cas12a protein can be modified to alter one or more properties (e.g., nuclease activity, affinity, stability, etc.) of the protein. Alternatively, domains of the Cas12a protein not involved in RNA targeting can be eliminated from the protein such that the modified Cas12a protein is smaller than the wild type Cas12a protein. In some embodiments, the present system utilizes the Cas12a protein from Acidaminococcus sp., either as encoded in bacteria or codon-optimized for expression in mammalian cells.

A mutant Cas protein refers to a polypeptide derivative of the wild type protein, e.g., a protein having one or more point mutations, insertions, deletions, truncations, a fusion protein, or a combination thereof. The mutant has at least one of the RNA-guided DNA binding activity, or RNA-guided nuclease activity, or both. In general, the modified version is at least 50% (e.g., any number between 50% and 100%, inclusive, e.g., 50%, 60%, 70%, 75%, 80%, 85%, 90%, 95%, and 99%) identical to the wild type protein (e.g., FnCpf1 from Francisella novicida, AsCpf1 from Acidaminococcus, or LbCpf1 from Lachnospiraceae).

In some embodiments, the Cas protein includes one or more conservative modifications. The Cas protein with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine).

In some embodiments, the Cas protein can be a chimeric protein containing a first fragment from a first Cas protein (e.g., Cas12a) ortholog and a second fragment from a second Cas protein (e.g., Cas12a) ortholog, wherein the first and second Cas protein orthologs are different. For example, the first and second Cas protein orthologs can be derived from different bacteria or archaea species, as described above.

In some embodiments, the Cas protein can be encoded by a codon-optimized sequence. For example, the nucleotide sequence encoding the Cas may be codon-optimized for expression in a eukaryote or eukaryotic cell. In some embodiments, the codon-optimized Cas protein is FnCpf1p, AsCpf1, or LbCpf1, which is codon-optimized for operability in a eukaryotic cell or organism, e.g., a yeast cell, or a mammalian cell or organism, including a mouse cell, a rat cell, and a human cell or non-human eukaryote organism (e.g., plant).

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.). In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codonusage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

b. crRNA and Corresponding Target Sequence

Due to its simplicity and efficiency, the CRISPR-Cas system has been used to perform genome-editing in cells of various organisms. The specificity of this system is dictated by base-pairing between a target sequence (e.g., target DNA sequence) and a crRNA sequence. Thus, the crRNA sequence provides the targeting specificity, which includes a region complementary and capable of hybridization to a pre-selected target site of interest. In some embodiments, a crRNA sequence can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the crRNA sequence is at least 16, 17, 18, 19, 20, 25 nucleotides, or between 16-30, or between 16-25, or between 16-20 nucleotides in length. In some embodiments, the crRNA sequence is 10-30 (e.g., 20-30) nucleotides in length.

In some embodiments, the system may include additional targeting sequence(s). For example, the system may include two or more targeting sequences, each of which comprises a guide nucleotide sequence encoding or comprising a crRNA sequence capable of hybridizing with a target sequence and forming a complex with the Cas protein. In some embodiments, the crRNA sequences contained in or encoded by the targeting sequences are different from one another. In some embodiments, the crRNA sequences hybridize with different target sequences.

The terms “crRNA,” “guide RNA,” “single guide RNA,” or “sgRNA” are used interchangeably as in PCT/US2013/074667. A crRNA sequence can be any polynucleotide sequence that has sufficient complementarity with a target polynucleotide sequence to hybridize with a target sequence and direct sequence-specific binding of a CRISPR complex to the target sequence. In some embodiments, the degree of complementarity between a crRNA sequence and its corresponding target sequence is about or more than about 50%, 60%, 75%, 80%, 85%, 90%, 95%, 97.5%, 99%, or more. Optimal alignment may be determined by a suitable sequence alignment algorithm. Examples of such a sequence alignment algorithm include the Smith-Waterman algorithm, the Needleman-Wunsch algorithm, algorithms based on the Burrows-Wheeler Aligner, CLUSTALW or CLUSTALX, BLAT, NOVOALIGN (NOVOCRAFT TECHNOLOGIES), ELAND (Illumina, San Diego, Calif.), SOAP (soap.genomics.org.cn), and MAQ (maq.sourceforge.net).

The ability of a crRNA sequence to direct sequence-specific binding of a CRISPR complex to a target sequence may be evaluated by any suitable assay known in the art, such as the Surveyor assay. For example, the described CRISPR-Cas system (e.g., Cas12a-based system) may be provided to a host cell having the corresponding target sequence, such as by transfection with vectors encoding the components of the CRISPR sequence, followed by an assessment of preferential cleavage within the target sequence by the Surveyor assay.

A crRNA sequence may be selected to target any target sequence. In some embodiments, the target sequence is a sequence within a genome of a cell, including those that are unique in the target genome. In some embodiments, a crRNA sequence is selected to reduce the degree of secondary structure within the crRNA. In some embodiments, about or less than about 75%, 50%, 40%, 30%, 25%, 20%, 15%, 10%, 5%, 1%, or fewer of the nucleotides of the crRNA participate in self-complementary base pairing when optimally folded. Optimal folding may be determined by any suitable polynucleotide folding algorithm, including the programs based on calculating the minimal Gibbs free energy, such as mFold (Nucleic Acids Res. 9 (1981), 133-148), RNAfold (see, e.g., A. R. Gruber et al., 2008, Cell 106(1): 23-24; and PA Carr and G M Church, 2009, Nature Biotechnology 27(12): 1151-62).

In the context of formation of a CRISPR complex, “target sequence” refers to a sequence to which a crRNA sequence is designed to target, e.g., have complementarity, where hybridization between a target sequence and a crRNA sequence promotes the formation of a CRISPR complex (e.g., Cas12a/crRNA complex). The section of the crRNA sequence through which complementarity to the target sequence is important for cleavage activity is referred to herein as the seed sequence. A target sequence may comprise any polynucleotide, such as DNA or RNA polynucleotides and is comprised within a target locus of interest. In some embodiments, a target sequence is located in the nucleus or cytoplasm of a cell. In some embodiments, the target sequence is within a cell, such as a eukaryotic cell. In some embodiments, the cell is a plant, animal, or human cell. In other embodiments, the target sequence is within virus or bacteria.

In some embodiments, the target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), microRNA (miRNA), small interfering RNA (siRNA), small nuclear RNA (snRNA), small nucleolar RNA (snoRNA), double-stranded RNA (dsRNA), non-coding RNA (ncRNA), long non-coding RNA (lncRNA), and small cytoplasmatic RNA (scRNA). In some embodiments, the target sequence may be a sequence within an RNA molecule selected from the group consisting of mRNA, pre-mRNA, and rRNA. In some embodiments, the target sequence may be a sequence within an RNA molecule selected from the group consisting of ncRNA and lncRNA. In some embodiments, the target sequence may be a sequence within an mRNA molecule or a pre-mRNA molecule.

One parameter for selecting a suitable target nucleic acid sequence is that it has a 5′ PAM site/sequence. Each target sequence and its corresponding PAM site/sequence are referred herein as a Cas-targeted site. In some embodiments, a PAM or PAM-like motif directs binding of the Cas protein complex to the target locus of interest. In some embodiments, the PAM is 5′ TTN, where N is A/C/G or T, and the Cas protein is FnCpf1p. In some embodiments, the PAM is 5′ TTTV, where V is A/C or G and the Cas protein is AsCpf1, LbCpf1, or PaCpf1p. In some embodiments, the PAM is 5′ TTN, where N is A/C/G or T, the Cas protein is FnCpf1p, and the PAM is located upstream of the 5′ end of the protospacer. In some embodiments, the PAM is 5′ CTA, where the Cas protein is FnCpf1p, and the PAM is located upstream of the 5′ end of the protospacer or the target locus. In some embodiments, this disclosure provides for an expanded targeting range for RNA guided genome editing nucleases wherein the T-rich PAMs of the Cpf1 family allow for targeting and editing of AT-rich genomes.

In some embodiments, the crRNA sequence comprises a nucleotide sequence of SEQ ID NOs: 20-29.

TABLE 2
Example crRNA Sequences

| | | SEQ |
|---|---|---|
| | | ID |
| Targeting Protein | Sequence | NO |
| beta-2 microglobulin 1 | UGGCCUGGAGGCUAUCCAGC | 20 |
| beta-2 microglobulin 2 | AUAUAAGUGGAGGCGUCGCG | 21 |
| beta-2 microglobulin 3 | CUCACGUCAUCCAGCAGAGA | 22 |
| CD47 molecule 1 | AUUAAAUAGUAGCUGAGCUG | 23 |
| | AUC | |
| CD47 molecule 2 | GCACUACUAAAGUCAGUGGG | 24 |
| | GAC | |
| CD47 molecule 3 | GUAAUGACACUGUCGUCAUU | 25 |
| | CCA | |
| Adhesion G Protein- | AGCAGGGCUUCCUCUGGAGC | 26 |
| Coupled Receptor E51 | UUC | |
| Adhesion G Protein- | UUGUGGUGCGCGUGUUCCAA | 27 |
| Coupled Receptor E52 | GGC | |
| Adhesion G Protein- | CUGGCCGCCUUCUGCUGGAU | 28 |
| Coupled Receptor E53 | GAG | |
| non targeting control | UCAUGCUUGCUUGGGCAAAA | 29 |
| (human) | | |

c. Direct Repeat

In some embodiments, a crRNA sequence may be linked to a direct repeat sequence. In some embodiments, the direct repeat sequence is located upstream (i.e., 5′) from the crRNA sequence. In some embodiments, the direct repeat sequence comprises one or more stem loops or optimized secondary structures. In some embodiments, the direct repeat has at least 16 nucleotides (e.g., 17, 18, 19, 20, 21, 22, 23, 24, 25 nucleotides) and optionally a single stem loop. In some embodiments, the direct repeat has more than one stem loop and optimized secondary structures. In some embodiments, the crRNA comprises a stem loop or an optimized stem loop structure or an optimized secondary structure, wherein the stem loop or optimized stem loop structure is important for cleavage activity. In some embodiments, the cleavage activity of the Cas-crRNA complex is modified by introducing mutations that affect the stem loop RNA duplex structure. In some embodiments, mutations which maintain the RNA duplex of the stem loop may be introduced, whereby the cleavage activity of the Cas protein complex is maintained.

In some embodiments, the direct repeat may include at least one protein-binding RNA aptamer, which may be included such as part of an optimized secondary structure. In some embodiments, the aptamer may be capable of binding a bacteriophage coat protein. The bacteriophage coat protein can be one of Qβ, F2, GA, fr, JP501, MS2, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, or PRR1.

In some embodiments, the direct repeat comprises a nucleotide sequence of SEQ ID NOs: 30-52.

TABLE 3
Example Direct Repeat Sequences

| | SEQ | |
|---|---|---|
| Direct Repeat | ID | |
| Sequence | NO | Note |
| AAUUUCUACUCUUGUAGAU | 30 | DR |
| GAUUUCUACUCUUGUAGAU | 31 | A18G |
| UUAAUCUACUCUUGUAGAU | 32 | UUAA |
| GUGACGACGGCUGUGGGCC | 33 | scrbl |
| AAUUUCUACUAAGUGUAGAU | 34 | Lachnospiraceae bacterium ND2006 |
| AAUUUCUACUAGUGUAGAU | 35 | Candidatus Methanomethylophilus |
| | | alvus |
| | | Mxl201 |
| AAUUUCUACUAUUGUAGAU | 36 | Sneatia amnii |
| AAUUUCUACUCUUGUAGAU | 37 | Acidaminococcus sp. BV3L6 |
| AAUUUCUACUUUUGUAGAU | 38 | Parcubacteria group bacterium |
| | | GW2011 |
| AAUUUCUACUUUUGUAGAU | 39 | Candidatus Roizmanbacteria |
| | | bacterium GW2011 |
| GAUUUCUACUUUUGUAGAU | 40 | Candidatus Peregrinbacterium |
| | | bacterium GW2011 |
| AAUUUCUACUAUUGUAGAU | 41 | Lachnospiracea bacterium MA2020 |
| AAUUUCUACUAUUGUAGAU | 42 | Butyrivibrio sp. NC3005 |
| AAUUUCUACUCUUGUAGAU | 43 | Butyrivibrio fibrisolvens |
| AAUUUCUACUAUUGUAGAU | 44 | Prevotella bryantii B14 |
| AAUUUCUACUAUUGUAGAU | 45 | Bacteroidetes oral taxon 274 |
| AAUUUCUACUAUUGUAGAU | 46 | Flavobacterium branchiophilum |
| | | FL-15 |
| AAUUUCUACUCUUGUAGAU | 47 | Lachnospiraceae bacterium MC2017 |
| AAUUUCUACUGUUUGUAGAU | 48 | Moraxella lacunata |
| AAUUUCUACUGUUUGUAGAU | 49 | Moraxella bovoculi AAX08_00205 |
| AAUUUCUACUGUUUGUAGAU | 50 | Moraxella bovoculi AAX11_00205 |
| AAUUUCUACUGUUGUAGAU | 51 | Francisella novicida U112 |
| AAUUUCUACUGUUGUAGAU | 52 | Thiomicrospira sp. XS5 |

d. microRNA-Target Site

As used herein, “microRNA” refers to any small RNA that can associate with the argonaute (AGO) family of proteins. microRNAs (or miRNAs) are small regulatory RNAs in the cell that can guide a microRNA-associated protein (e.g., endonuclease, such as Ago2) to the microRNA-target site, a sequence complementary to the microRNA, resulting in cleavage of the microRNA-target site. The microRNA expression profile varies between cell-types, and there are numerous microRNAs unique to each cell-type. In some embodiments, to confer cell-specificity to the aforementioned CRISPR-Cas systems disclosed herein, a microRNA-target site can be introduced downstream (or 3′) of the crRNA sequence, followed by an AU-rich element (ARE) and/or another destabilizing element (or referred to as degradation tag), such as a ribozyme which would remove the poly-A tail and induce RNA degradation (FIG. 8). AREs can cause destabilization and rapid degradation of the RNA. The microRNA will guide a microRNA-associated protein (e.g., Ago2) to the microRNA-target site and cleave off the destabilizing ARE, thus leaving a functional crRNA intact only when a given microRNA (e.g., microRNA-106) is present in cells.

microRNAs interact with various microRNA-associated proteins. For example, microRNAs interact with members of the RISC (RNA-induced silencing complex) pathway to suppress translation of one or more messenger RNAs (e.g., microRNA-target site). Ago2 (also known in the art as Argonaute 2 and EIF2C2) is the only component of the RISC pathway with known RNAse activity in human cells. In certain instances, Ago2 binds to a microRNA, which in turn hybridizes with a region of a microRNA-target site that is at least partially complementary to a portion of the microRNA.

In some embodiments, a microRNA has a nucleobase sequence as set forth in miRBase, a database of published microRNA sequences found at http://microrna.sanger.ac.uk/sequences/. In certain embodiments, a microRNA has a nucleobase sequence as set forth in miRBase version 18.0 released November 2011, which is herein incorporated by reference in its entirety.

As used herein, “microRNA-associated protein” refers to a protein that interacts directly with a microRNA. In some embodiments, the miroRNA-associated protein is a RISC protein. In some embodiments, the miroRNA-associated protein is Ago2.

In some embodiments, the microRNA-target site is at least partially complementary to a portion of a microRNA in cells. In some embodiments, the CRISPR-Cas systems disclosed herein comprises more than one microRNA-target sites, which enable crRNA activation in more than one tissue. For example, to build a construct that works in both neurons and microglial cells, the construct may include both miR-124 (neuronal) and miR-142 (microglial) targets.

In some embodiments, the microRNA-target site is selected from the group consisting of SEQ ID NOs: 199-344.

TABLE 4
Example microRNA-target Site Sequences

| | | SEQ | | SEQ |
|---|---|---|---|---|
| | microRNA | ID | microRNA-target | ID |
| microRNA ID | sequence | NO | Site Sequence | NO |
| hsa-let-7a-2-3p MIMAT0010195 | CUGUACAGCCUC | 53 | GGAAAGCTAGG | 199 |
| Homo sapiens let-7a-2-3p | CUAGCUUUCC | | AGGCTGTACAG | |
| hsa-miR-1-3p MIMAT0000416 | UGGAAUGUAAA | 54 | ATACATACTTCT | 200 |
| Homo sapiens miR-1-3p | GAAGUAUGUAU | | TTACATTCCA | |
| hsa-miR-100-3p MIMAT0004512 | CAAGCUUGUAUC | 55 | CATACCTATAGA | 201 |
| Homo sapiens miR-100-3p | UAUAGGUAUG | | TACAAGCTTG | |
| hsa-miR- 103a-1+10-5p | GGCUUCUUUACA | 56 | CAAGGCAGCAC | 202 |
| MIMAT0037306 Homo sapiens | GUGCUGCCUUG | | TGTAAAGAAGC | |
| mir-103a-1-5p | | | C | |
| hsa-miR-105-3p MIMAT0004516 | ACGGAUGUUUG | 57 | TAGCACATGCTC | 203 |
| Homo sapiens miR-105-3p | AGCAUGUGCUA | | AAACATCCGT | |
| hsa-miR-106a-3p MIMAT0004517 | CUGCAAUGUAAG | 58 | GTAAGAAGTGC | 204 |
| Homo sapiens miR-106a-3p | CACUUCUUAC | | TTACATTGCAG | |
| hsa-miR-106a-5p MIMAT0000103 | AAAAGUGCUUAC | 59 | CTACCTGCACTG | 205 |
| Homo sapiens miR-106a-5p | AGUGCAGGUAG | | TAAGCACTTTT | |
| hsa-miR-106b-3p MIMAT0004672 | CCGCACUGUGGG | 60 | GCAGCAAGTAC | 206 |
| Homo sapiens miR-106b-3p | UACUUGCUGC | | CCACAGTGCGG | |
| hsa-miR-106b-5p MIMAT0000680 | UAAAGUGCUGAC | 61 | ATCTGCACTGTC | 207 |
| Homo sapiens miR-106b-5p | AGUGCAGAU | | AGCACTTTA | |
| hsa-miR-13 0a-3p MIMAT0000425 | CAGUGCAAUGUU | 62 | ATGCCCTTTTAA | 208 |
| Homo sapiens miR-130a-3p | AAAAGGGCAU | | CATTGCACTG | |
| hsa-miR-13 0a-5p MIMAT0004593 | GCUCUUUUCACA | 63 | AGTAGCACAAT | 209 |
| Homo sapiens miR-130a-5p | UUGUGCUACU | | GTGAAAAGAGC | |
| hsa-miR-133a-3p MIMAT0000427 | UUUGGUCCCCUU | 64 | CAGCTGGTTGAA | 210 |
| Homo sapiens miR-133a-3p | CAACCAGCUG | | GGGGACCAAA | |
| hsa-miR-133a-5p MIMAT0026478 | AGCUGGUAAAA | 65 | ATTTGGTTCCAT | 211 |
| Homo sapiens miR-133a-5p | UGGAACCAAAU | | TTTACCAGCT | |
| hsa-miR-135a-5p MIMAT0000428 | UAUGGCUUUUU | 66 | TCACATAGGAAT | 212 |
| Homo sapiens miR-135a-5p | AUUCCUAUGUGA | | AAAAAGCCATA | |
| hsa-miR-142-3p MIMAT0000434 | UGUAGUGUUUCC | 67 | TCCATAAAGTAG | 213 |
| Homo sapiens miR-142-3p | UACUUUAUGGA | | GAAACACTACA | |
| hsa-miR-142-5p MIMAT0000433 | CAUAAAGUAGA | 68 | AGTAGTGCTTTC | 214 |
| Homo sapiens miR-142-5p | AAGCACUACU | | TACTTTATG | |
| hsa-miR-155-3p MIMAT0004658 | CUCCUACAUAUU | 69 | TGTTAATGCTAA | 215 |
| Homo sapiens miR-155-3p | AGCAUUAACA | | TATGTAGGAG | |
| hsa-miR-15a-3p MIMAT0004488 | CAGGCCAUAUUG | 70 | TGAGGCAGCAC | 216 |
| Homo sapiens miR-15a-3p | UGCUGCCUCA | | AATATGGCCTG | |
| hsa-miR-16-5p MIMAT0000069 | UAGCAGCACGUA | 71 | CGCCAATATTTA | 217 |
| Homo sapiens miR-16-5p | AAUAUUGGCG | | CGTGCTGCTA | |
| hsa-miR-17-3p MIMAT0000071 | ACUGCAGUGAAG | 72 | CTACAAGTGCCT | 218 |
| Homo sapiens miR-17-3p | GCACUUGUAG | | TCACTGCAGT | |
| hsa-miR-181a-2-3p | ACCACUGACCGU | 73 | GGTACAGTCAA | 219 |
| MIMAT0004558 Homo sapiens | UGACUGUACC | | CGGTCAGTGGT | |
| mir-181a-2-3p | | | | |
| hsa-miR-20a-3p MIMAT0004493 | ACUGCAUUAUGA | 74 | CTTTAAGTGCTC | 220 |
| Homo sapiens miR-20a-3p | GCACUUAAAG | | ATAATGCAGT | |
| hsa-miR-21-3p MIMAT0004494 | CAACACCAGUCG | 75 | ACAGCCCATCG | 221 |
| Homo sapiens miR-21-3p | AUGGGCUGU | | ACTGGTGTTG | |
| hsa-miR-22-5p MIMAT0004495 | AGUUCUUCAGUG | 76 | TAAAGCTTGCCA | 222 |
| Homo sapiens miR-22-5p | GCAAGCUUUA | | CTGAAGAACT | |
| hsa-miR-221-3p MIMAT0000278 | AGCUACAUUGUC | 77 | GAAACCCAGCA | 223 |
| Homo sapiens miR-221+10-3p | UGCUGGGUUUC | | GACAATGTAGCT | |
| hsa-miR-221-5p MIMAT0004568 | ACCUGGCAUACA | 78 | AAATCTACATTG | 224 |
| Homo sapiens miR-221+10-5p | AUGUAGAUUU | | TATGCCAGGT | |
| hsa-miR-23a-3p MIMAT0000078 | AUCACAUUGCCA | 79 | GGAAATCCCTG | 225 |
| Homo sapiens miR-23a-3p | GGGAUUUCC | | GCAATGTGAT | |
| hsa-miR-26a-1+10-3 p | CCUAUUCUUGGU | 80 | CGTGCAAGTAA | 226 |
| MIMAT0004499 Homo sapiens | UACUUGCACG | | CCAAGAATAGG | |
| mir-26a-1-3p | | | | |
| hsa-miR-29b-1+10-5 p | GCUGGUUUCAUA | 81 | TCTAAACCACCA | 227 |
| MIMAT0004514 Homo sapiens | UGGUGGUUUAG | | TATGAAACCAG | |
| mir-29b-1-5p | A | | C | |
| hsa-miR-300 MIMAT0004903 | UAUACAAGGGCA | 82 | AGAGAGAGTCT | 228 |
| Homo sapiens miR-300 | GACUCUCUCU | | GCCCTTGTATA | |
| hsa-miR-302d-5p MIMAT0004685 | ACUUUAACAUGG | 83 | GCAAGTGCCTCC | 229 |
| Homo sapiens miR-302d-5p | AGGCACUUGC | | ATGTTAAAGT | |
| hsa-miR-30a-3p MIMAT0000088 | CUUUCAGUCGGA | 84 | GCTGCAAACATC | 230 |
| Homo sapiens miR-30a-3p | UGUUUGCAGC | | CGACTGAAAG | |
| hsa-miR-31-3p MIMAT0004504 | UGCUAUGCCAAC | 85 | ATGGCAATATGT | 231 |
| Homo sapiens miR-31+10-3p | AUAUUGCCAU | | TGGCATAGCA | |
| hsa-miR-32-3p MIMAT0004505 | CAAUUUAGUGU | 86 | AAATATCACAC | 232 |
| Homo sapiens miR-32-3p | GUGUGAUAUUU | | ACACTAAATTG | |
| hsa-miR-320e MIMATOO15072 | AAAGCUGGGUU | 87 | CCTTCTCAACCC | 233 |
| Homo sapiens miR-320e | GAGAAGG | | AGCTTT | |
| hsa-miR-323a-3p MIMAT0000755 | CACAUUACACGG | 88 | AGAGGTCGACC | 234 |
| Homo sapiens miR-323a-3p | UCGACCUCU | | GTGTAATGTG | |
| hsa-miR-324-3p MIMAT0000762 | CCCACUGCCCCA | 89 | CCAGCAGCACCT | 235 |
| Homo sapiens miR-324-3p | GGUGCUGCUGG | | GGGGCAGTGGG | |
| hsa-miR-325 MIMAT0000771 | CCUAGUAGGUGU | 90 | ACACTTACTGGA | 236 |
| Homo sapiens miR-325 | CCAGUAAGUGU | | CACCTACTAGG | |
| hsa-miR-328-5p MIMAT0026486 | GGGGGGGCAGG | 91 | CCCTGAGCCCCT | 237 |
| Homo sapiens miR-328-5p | AGGGGCUCAGGG | | CCTGCCCCCCC | |
| hsa-miR-329-5p MIMAT0026555 | GAGGUUUUCUG | 92 | GAAACAGAAAC | 238 |
| Homo sapiens miR-329-5p | GGUUUCUGUUUC | | CCAGAAAACCT | |
| | | | C | |
| hsa-miR-330-5p MIMAT0004693 | UCUCUGGGCCUG | 93 | GCCTAAGACAC | 239 |
| Homo sapiens miR-330-5p | UGUCUUAGGC | | AGGCCCAGAGA | |
| hsa-miR-331+10-3p MIMAT0000760 | GCCCCUGGGCCU | 94 | TTCTAGGATAGG | 240 |
| Homo sapiens miR-331-3p | AUCCUAGAA | | CCCAGGGGC | |
| hsa-miR-335-3p MIMAT0004703 | UUUUUCAUUAU | 95 | GGTCAGGAGCA | 241 |
| Homo sapiens miR-335-3p | UGCUCCUGACC | | ATAATGAAAAA | |
| hsa-miR-337-5p MIMAT0004695 | GAACGGCUUCAU | 96 | AACTCCTGTATG | 242 |
| Homo sapiens miR-337-5p | ACAGGAGUU | | AAGCCGTTC | |
| hsa-miR-338-3p MIMAT0000763 | UCCAGCAUCAGU | 97 | CAACAAAATCA | 243 |
| Homo sapiens miR-338-3p | GAUUUUGUUG | | CTGATGCTGGA | |
| hsa-miR-339-5p MIMAT0000764 | UCCCUGUCCUCC | 98 | CGTGAGCTCCTG | 244 |
| Homo sapiens miR-339-5p | AGGAGCUCACG | | GAGGACAGGGA | |
| hsa-miR-33a-3p MIMAT0004506 | CAAUGUUUCCAC | 99 | GTGATGCACTGT | 245 |
| Homo sapiens miR-33a-3p | AGUGCAUCAC | | GGAAACATTG | |
| hsa-miR-340-5p MIMAT0004692 | UUAUAAAGCAA | 100 | AATCAGTCTCAT | 246 |
| Homo sapiens miR-340-5p | UGAGACUGAUU | | TGCTTTATAA | |
| hsa-miR-342-3p MIMAT0000753 | UCUCACACAGAA | 101 | ACGGGTGCGATT | 247 |
| Homo sapiens miR-342-3p | AUCGCACCCGU | | TCTGTGTGAGA | |
| hsa-miR-345-5p MIMAT0000772 | GCUGACUCCUAG | 102 | GAGCCCTGGACT | 248 |
| Homo sapiens miR-345-5p | UCCAGGGCUC | | AGGAGTCAGC | |
| hsa-miR-346 MIMAT0000773 | UGUCUGCCCGCA | 103 | AGAGGCAGGCA | 249 |
| Homo sapiens miR-346 | UGCCUGCCUCU | | TGCGGGCAGAC | |
| | | | A | |
| hsa-miR-34a-3p MIMAT0004557 | CAAUCAGCAAGU | 104 | AGGGCAGTATA | 250 |
| Homo sapiens miR-34a-3p | AUACUGCCCU | | CTTGCTGATTG | |
| hsa-miR-519a-2-5p | CCUCUACAGGGA | 105 | GAAAGCGCTTCC | 251 |
| MIMAT0037327 Homo sapiens | AGCGCUUUC | | CTGTAGAGG | |
| mir-519a-2-5p | | | | |
| hsa-miR-520a-3p MIMAT0002834 | AAAGUGCUUCCC | 106 | ACAGTCCAAAG | 252 |
| Homo sapiens miR-520a-3p | UUUGGACUGU | | GGAAGCACTTT | |
| hsa-miR-524-5p MIMAT0002849 | CUACAAAGGGAA | 107 | GAGAAAGTGCT | 253 |
| Homo sapiens miR-524-5p | GCACUUUCUC | | TCCCTTTGTAG | |
| hsa-miR-525-3p MIMAT0002839 | GAAGGCGCUUCC | 108 | CGCTCTAAAGG | 254 |
| Homo sapiens miR-525-3p | CUUUAGAGCG | | GAAGCGCCTTC | |
| hsa-miR-561-3p MIMAT0003225 | CAAAGUUUAAG | 109 | ACTTCAAGGATC | 255 |
| Homo sapiens miR-561-3p | AUCCUUGAAGU | | TTAAACTTTG | |
| hsa-miR-571 MIMAT0003236 | UGAGUUGGCCAU | 110 | CTCACTCAGATG | 256 |
| Homo sapiens miR-571 | CUGAGUGAG | | GCCAACTCA | |
| hsa-miR-572 MIMAT0003237 | GUCCGCUCGGCG | 111 | TGGGCCACCGCC | 257 |
| Homo sapiens miR-572 | GUGGCCCA | | GAGCGGAC | |
| hsa-miR-573 MIMAT0003238 | CUGAAGUGAUG | 112 | CTGATCAGTTAC | 258 |
| Homo sapiens miR-573 | UGUAACUGAUCA | | ACATCACTTCAG | |
| | G | | | |
| hsa-miR-600 MIMAT0003268 | ACUUACAGACAA | 113 | GAGCAAGGCTC | 259 |
| Homo sapiens miR-600 | GAGCCUUGCUC | | TTGTCTGTAAGT | |
| hsa-miR-601 MIMAT0003269 | UGGUCUAGGAU | 114 | CTCCTCCAACAA | 260 |
| Homo sapiens miR-601 | UGUUGGAGGAG | | TCCTAGACCA | |
| hsa-miR-602 MIMAT0003270 | GACACGGGCGAC | 115 | GGGCCGCAGCT | 261 |
| Homo sapiens miR-602 | AGCUGCGGCCC | | GTCGCCCGTGTC | |
| hsa-miR-603 MIMAT0003271 | CACACACUGCAA | 116 | GCAAAAGTAAT | 262 |
| Homo sapiens miR-603 | UUACUUUUGC | | TGCAGTGTGTG | |
| hsa-miR-604 MIMAT0003272 | AGGCUGCGGAAU | 117 | GTCCTGAATTCC | 263 |
| Homo sapiens miR-604 | UCAGGAC | | GCAGCCT | |
| hsa-miR-605-3p MIMAT0026621 | AGAAGGCACUAU | 118 | TCTAAATCTCAT | 264 |
| Homo sapiens miR-605-3p | GAGAUUUAGA | | AGTGCCTTCT | |
| hsa-miR-628-5p MIMAT0004809 | AUGCUGACAUAU | 119 | CCTCTAGTAAAT | 265 |
| Homo sapiens miR-628-5p | UUACUAGAGG | | ATGTCAGCAT | |
| hsa-miR-629-3p MIMAT0003298 | GUUCUCCCAACG | 120 | GCTGGGCTTACG | 266 |
| Homo sapiens miR-629-3p | UAAGCCCAGC | | TTGGGAGAAC | |
| hsa-miR-629-5p MIMAT0004810 | UGGGUUUACGU | 121 | AGTTCTCCCAAC | 267 |
| Homo sapiens miR-629-5p | UGGGAGAACU | | GTAAACCCA | |
| hsa-miR-630 MIMAT0003299 | AGUAUUCUGUAC | 122 | ACCTTCCCTGGT | 268 |
| Homo sapiens miR-630 | CAGGGAAGGU | | ACAGAATACT | |
| hsa-miR-631 MIMAT0003300 | AGACCUGGCCCA | 123 | GCTGAGGTCTGG | 269 |
| Homo sapiens miR-631 | GACCUCAGC | | GCCAGGTCT | |
| hsa-miR-632 MIMAT0003302 | GUGUCUGCUUCC | 124 | TCCCACAGGAA | 270 |
| Homo sapiens miR-63 2 | UGUGGGA | | GCAGACAC | |
| hsa-miR-633 MIMAT0003303 | CUAAUAGUAUCU | 125 | TTTATTGTGGTA | 271 |
| Homo sapiens miR-63 3 | ACCACAAUAAA | | GATACTATTAG | |
| hsa-miR-634 MIMAT0003304 | AACCAGCACCCC | 126 | GTCCAAAGTTGG | 272 |
| Homo sapiens miR-63 4 | AACUUUGGAC | | GGTGCTGGTT | |
| hsa-miR-635 MIMAT0003305 | ACUUGGGCACUG | 127 | GGACATTGTTTC | 273 |
| Homo sapiens miR-63 5 | AAACAAUGUCC | | AGTGCCCAAGT | |
| hsa-miR-636 MIMAT0003306 | UGUGCUUGCUCG | 128 | TGCGGGCGGGA | 274 |
| Homo sapiens miR-63 6 | UCCCGCCCGCA | | CGAGCAAGCAC | |
| | | | A | |
| hsa-miR-637 MIMAT0003307 | ACUGGGGGCUUU | 129 | ACGCAGAGCCC | 275 |
| Homo sapiens miR-63 7 | CGGGCUCUGCGU | | GAAAGCCCCCA | |
| | | | GT | |
| hsa-miR-638 MIMAT0003308 | AGGGAUCGCGGG | 130 | AGGCCGCCACC | 276 |
| Homo sapiens miR-63 8 | CGGGUGGCGGCC | | CGCCCGCGATCC | |
| | U | | CT | |
| hsa-miR-639 MIMAT0003309 | AUCGCUGCGGUU | 131 | ACAGCGCTCGC | 277 |
| Homo sapiens miR-63 9 | GCGAGCGCUGU | | AACCGCAGCGA | |
| | | | T | |
| hsa-miR-640 MIMAT0003310 | AUGAUCCAGGAA | 132 | AGAGGCAGGTT | 278 |
| Homo sapiens miR-640 | CCUGCCUCU | | CCTGGATCAT | |
| hsa-miR-641 MIMAT0003311 | AAAGACAUAGG | 133 | GAGGTGACTCTA | 279 |
| Homo sapiens miR-641 | AUAGAGUCACCU | | TCCTATGTCTTT | |
| | C | | | |
| hsa-miR-9-3p MIMAT0000442 | AUAAAGCUAGA | 134 | ACTTTCGGTTAT | 280 |
| Homo sapiens miR-9-3p | UAACCGAAAGU | | CTAGCTTTAT | |
| hsa-miR-92a-1+10-5 p | AGGUUGGGAUC | 135 | AGCATTGCAACC | 281 |
| MIMAT0004507 Homo sapiens | GGUUGCAAUGCU | | GATCCCAACCT | |
| mir-92a-1-5p | | | | |
| hsa-miR-92a-2-5p | GGGUGGGGAUU | 136 | GTAATGCAACA | 282 |
| MIMAT0004508 Homo sapiens | UGUUGCAUUAC | | AATCCCCACCC | |
| mir-92a-2-5p | | | | |
| hsa-miR-93-5p MIMAT0000093 | CAAAGUGCUGUU | 137 | CTACCTGCACGA | 283 |
| Homo sapiens miR-93-5p | CGUGCAGGUAG | | ACAGCACTTTG | |
| hsa-miR-95-3p MIMAT0000094 | UUCAACGGGUAU | 138 | TGCTCAATAAAT | 284 |
| Homo sapiens miR-95-3p | UUAUUGAGCA | | ACCCGTTGAA | |
| hsa-miR-96-5p MIMAT0000095 | UUUGGCACUAGC | 139 | AGCAAAAATGT | 285 |
| Homo sapiens miR-96-5p | ACAUUUUUGCU | | GCTAGTGCCAA | |
| | | | A | |
| hsa-miR-98-5p MIMAT0000096 | UGAGGUAGUAA | 140 | AACAATACAAC | 286 |
| Homo sapiens miR-98-5p | GUUGUAUUGUU | | TTACTACCTCA | |
| hsa-miR-99a-3p MIMAT0004511 | CAAGCUCGCUUC | 141 | CAGACCCATAG | 287 |
| Homo sapiens miR-99a-3p | UAUGGGUCUG | | AAGCGAGCTTG | |
| abu-miR-106 MIMAT0042143 | UAAAGUGCUUAC | 142 | CTACCTGCACTG | 288 |
| Astatotilapia burtoni miR-106 | AGUGCAGGUAG | | TAAGCACTTTA | |
| abu-miR-142-3p MIMAT0042104 | GUAGUGUUUCCU | 143 | CCATAAAGTAG | 289 |
| Astatotilapia burtoni miR-142-3p | ACUUUAUGG | | GAAACACTAC | |
| aca-miR-142-3p MIMAT0021768 | UGUAGUGUUUCC | 144 | CATAAAGTAGG | 290 |
| Anolis carolinensis miR-142-3p | UACUUUAUG | | AAACACTACA | |
| aja-miR-142 MIMAT0031136 | UGUAGUGUUUCC | 145 | TCCATAAAGTAG | 291 |
| Artibeus jamaicensis miR-142 | UACUUUAUGGA | | GAAACACTACA | |
| ami-miR-142-3p MIMAT0038163 | UGUAGUGUUUCC | 146 | TCCATAAAGTAG | 292 |
| Alligator mississippiensis miR- | UACUUUAUGGA | | GAAACACTACA | |
| 142-3p | | | | |
| bta-miR-106a MIMAT0003784 | AAAAGUGCUUAC | 147 | TACCTGCACTGT | 293 |
| Bos taurus miR-106a | AGUGCAGGUA | | AAGCACTTTT | |
| bta-miR-142-3p MIMAT0003791 | AGUGUUUCCUAC | 148 | CATCCATAAAGT | 294 |
| Bos taurus miR-142-3p | UUUAUGGAUG | | AGGAAACACT | |
| ccr-miR-142-3p MIMAT0026223 | GUAGUGUUUCCU | 149 | CCATAAAGTAG | 295 |
| Cyprinus carpio miR-142-3p | ACUUUAUGG | | GAAACACTAC | |
| cgr-miR-142-3p MIMAT0023771 | GUAGUGUUUCCU | 150 | CCATAAAGTAG | 296 |
| Cricetulus griseus miR-142-3p | ACUUUAUGG | | GAAACACTAC | |
| cja-miR-142 MIMAT0039560 | GUAGUGUUUCCU | 151 | TCCATAAAGTAG | 297 |
| Callithrix jacchus miR-142 | ACUUUAUGGA | | GAAACACTAC | |
| cli-miR-106-5p MIMAT0038487 | AAAAGUGCUUAC | 152 | CTACCTGCACTG | 298 |
| Columba livia miR-106-5p | AGUGCAGGUAG | | TAAGCACTTTT | |
| cli-miR-142-3p MIMAT0038530 | UAGUGUUUCCUA | 153 | TCCATAAAGTAG | 299 |
| Columba livia miR-142-3p | CUUUAUGGA | | GAAACACTA | |
| cpi-miR-106a-5p MIMAT0037721 | AAAAGUGCUUAC | 154 | CTACCTGCACTG | 300 |
| Chrysemys picta miR-106a-5p | AGUGCAGGUAG | | TAAGCACTTTT | |
| cpi-miR-142-3p MIMAT0037767 | UGUAGUGUUUCC | 155 | CCATAAAGTAG | 301 |
| Chrysemys picta miR-142-3p | UACUUUAUGG | | GAAACACTACA | |
| cpo-miR-106a-5p MIMAT0046955 | AAAAGUGCUUAC | 156 | CTACCTGCACTG | 302 |
| Cavia porcellus miR-106a-5p | AGUGCAGGUAG | | TAAGCACTTTT | |
| cpo-miR-142-3p MIMAT0047009 | UAGUGUUUCCUA | 157 | TCCATAAAGTAG | 303 |
| Cavia porcellus miR-142-3 p | CUUUAUGGA | | GAAACACTA | |
| dno-miR-142-3p MIMAT0047658 | UAGUGUUUCCUA | 158 | TCCATAAAGTAG | 304 |
| Dasypus novemcinctus miR-142- | CUUUAUGGA | | GAAACACTA | |
| 3p | | | | |
| dre-miR-142a-3p MIMAT0003160 | UGUAGUGUUUCC | 159 | TCCATAAAGTAG | 305 |
| Danio rerio miR-142a-3p | UACUUUAUGGA | | GAAACACTACA | |
| eca-miR-142-3p MIMAT0013023 | UGUAGUGUUUCC | 160 | TCCATAAAGTAG | 306 |
| Equus caballus miR-142-3p | UACUUUAUGGA | | GAAACACTACA | |
| efu-miR-142 MIMAT0035200 | UGUAGUGUUUCC | 161 | TCCATAAAGTAG | 307 |
| Eptesicus fuscus miR-142 | UACUUUAUGGA | | GAAACACTACA | |
| gga-miR-106-5p MIMAT0001142 | AAAAGUGCUUAC | 162 | TACCTGCACTGT | 308 |
| Gallus gallus miR-106-5p | AGUGCAGGUA | | AAGCACTTTT | |
| gga-miR-142-3p MIMAT0001194 | UGUAGUGUUUCC | 163 | CCATAAAGTAG | 309 |
| Gallus gallus miR-142-3p | UACUUUAUGG | | GAAACACTACA | |
| gmo-miR-142+10-2+10-3p | UGUAGUGUUUCC | 164 | CCATAAAGTAG | 310 |
| MIMAT0044046 Gadus morhua | UACUUUAUGG | | GAAACACTACA | |
| mir-142-2-3p | | | | |
| hsa-miR-106a-5p MIMAT0000103 | AAAAGUGCUUAC | 165 | CTACCTGCACTG | 311 |
| Homo sapiens miR-106a-5p | AGUGCAGGUAG | | TAAGCACTTTT | |
| hsvl-miR-Hl-3p MIMAT0015220 | UACACCCCCCUG | 166 | AGGGTGGAAGG | 312 |
| Herpes Simplex miR-Hl-3p | CCUUCCACCCU | | CAGGGGGGTGT | |
| | | | A | |
| ipu-miR-142 MIMAT0029419 | GUAGUGUUUCCU | 167 | TCCATAAAGTAG | 313 |
| Ictalurus punctatus miR-142 | ACUUUAUGGA | | GAAACACTAC | |
| mdo-miR-142 MIMAT0004112 | UGUAGUGUUUCC | 168 | TCCATAAAGTAG | 314 |
| Monodelphis domestica miR-142 | UACUUUAUGGA | | GAAACACTACA | |
| mml-miR-142-3p MIMAT0006200 | UGUAGUGUUUCC | 169 | TCCATAAAGTAG | 315 |
| Macaca mulatta miR-142-3p | UACUUUAUGGA | | GAAACACTACA | |
| mmu-miR-106a-5p | CAAAGUGCUAAC | 170 | CTACCTGCACTG | 316 |
| MIMAT0000385 Mus musculus | AGUGCAGGUAG | | TTAGCACTTTG | |
| mir-106a-5p | | | | |
| mmu-miR-142a-3p | UGUAGUGUUUCC | 171 | TCCATAAAGTAG | 317 |
| MIMAT0000155 Mus musculus | UACUUUAUGGA | | GAAACACTACA | |
| mir-142a-3p | | | | |
| mze-miR-1 MIMAT0042359 | UGGAAUGUAAA | 172 | ATACATACTTCT | 318 |
| Metriaclima zebra miR-1 | GAAGUAUGUAU | | TTACATTCCA | |
| mze-miR-106 MIMAT0042366 | UAAAGUGCUUAC | 173 | CTACCTGCACTG | 319 |
| Metriaclima zebra miR-106 | AGUGCAGGUAG | | TAAGCACTTTA | |
| oan-miR-106-5p MIMAT0006849 | AAAAGUGCUUAC | 174 | ACCTGCACTGTA | 320 |
| Ornithorhynchus anatinus miR- | AGUGCAGGU | | AGCACTTTT | |
| 106-5p | | | | |
| oan-miR-142-3p MIMAT0006983 | UGUAGUGUUUCC | 175 | CCATAAAGTAG | 321 |
| Ornithorhynchus anatinus miR- | UACUUUAUGG | | GAAACACTACA | |
| 142-3p | | | | |
| oan-miR-142-3p MIMAT0006983 | UGUAGUGUUUCC | 176 | CCATAAAGTAG | 322 |
| Ornithorhynchus anatinus miR- | UACUUUAUGG | | GAAACACTACA | |
| 142-3p | | | | |
| oar-miR-106a MIMAT0030061 | AAAAGUGCUUAC | 177 | ACCTGCACTGTA | 323 |
| Ovis aries miR-106a | AGUGCAGGU | | AGCACTTTT | |
| ocu-miR-106a-5p MIMAT0048195 | AAAAGUGCUUAC | 178 | CTACCTGCACTG | 324 |
| Oryctolagus cuniculus miR-106a- | AGUGCAGGUAG | | TAAGCACTTTT | |
| 5p | | | | |
| ocu-miR-142-3p MIMAT0048246 | UAGUGUUUCCUA | 179 | TCCATAAAGTAG | 325 |
| Oryctolagus cuniculus miR-142-3p | CUUUAUGGA | | GAAACACTA | |
| oha-miR-142-3p MIMAT0036722 | UGUAGUGUUUCC | 180 | TCCATAAAGTAG | 326 |
| Ophiophagus hannah miR-142-3p | UACUUUAUGGA | | GAAACACTACA | |
| oni-miR-106 MIMAT0042734 | UAAAGUGCUUAC | 181 | CTACCTGCACTG | 327 |
| Oreochromis niloticus miR-106 | AGUGCAGGUAG | | TAAGCACTTTA | |
| pal-miR-106a-5p MIMAT0040204 | AAAAGUGCUUAC | 182 | CTACCTGCACTG | 328 |
| Pteropus alecto miR-106a-5p | AGUGCAGGUAG | | TAAGCACTTTT | |
| pal-miR-142-3p MIMAT0040131 | AGUGUUUCCUAC | 183 | CATCCATAAAGT | 329 |
| Pteropus alecto miR-142-3 p | UUUAUGGAUG | | AGGAAACACT | |
| pbv-miR-142-3p MIMAT0038954 | UAGUGUUUCCUA | 184 | TCCATAAAGTAG | 330 |
| Python bivittatus miR-142-3p | CUUUAUGGA | | GAAACACTA | |
| pny-miR-106 MIMAT0042920 | UAAAGUGCUUAC | 185 | CTACCTGCACTG | 331 |
| Pundamilia nyererei miR-106 | AGUGCAGGUAG | | TAAGCACTTTA | |
| ppy-miR-142-3p MIMAT0015766 | UGUAGUGUUUCC | 186 | TCCATAAAGTAG | 332 |
| Pongo pygmaeus miR-142-3p | UACUUUAUGGA | | GAAACACTACA | |
| ptr-miR-142 MIMAT0008037 Pan | UGUAGUGUUUCC | 187 | TCCATAAAGTAG | 333 |
| troglodytes miR-142 | UACUUUAUGGA | | GAAACACTACA | |
| mo-miR-142-3p MIMAT0000848 | UGUAGUGUUUCC | 188 | TCCATAAAGTAG | 334 |
| Rattus norvegicus miR-142-3p | UACUUUAUGGA | | GAAACACTACA | |
| ssa-miR-106a-5p MIMAT0032286 | UAAAGUGCUUAC | 189 | TACCTGCACTGT | 335 |
| Salmo salar miR-106a-5p | AGUGCAGGUA | | AAGCACTTTA | |
| ssa-miR-106b-5p MIMAT0032288 | AAAAGUGCUUAC | 190 | TACCTGCACTGT | 336 |
| Salmo salar miR-106b-5p | AGUGCAGGUA | | AAGCACTTTT | |
| ssa-miR-142a-3p MIMAT0032362 | AGUGUUUCCUAC | 191 | CATCCATAAAGT | 337 |
| Salmo salar miR-142a-3p | UUUAUGGAUG | | AGGAAACACT | |
| ssa-miR-142b-3p MIMAT0032364 | UGUAGUGUUUCC | 192 | ATCCATAAAGTA | 338 |
| Salmo salar miR-142b-3p | UACUUUAUGGA | | GGAAACACTAC | |
| | U | | A | |
| ssc-miR-142-3p MIMAT0020362 | UGUAGUGUUUCC | 193 | CCATAAAGTAG | 339 |
| Sus scrofa miR-142-3p | UACUUUAUGG | | GAAACACTACA | |
| tch-miR-142 MIMAT0036470 | GUAGUGUUUCCU | 194 | TCCATAAAGTAG | 340 |
| Tupaia chinensis miR-142 | ACUUUAUGGA | | GAAACACTAC | |
| tgu-miR-106-5p MIMATOO14590 | AAAAGUGCUUAC | 195 | CTACCTGCACTG | 341 |
| Taeniopygia guttata miR-106-5p | AGUGCAGGUAG | | TAAGCACTTTT | |
| tgu-miR-142-3p MIMAT0026999 | GUAGUGUUUCCU | 196 | TCCATAAAGTAG | 342 |
| Taeniopygia guttata miR-142-3p | ACUUUAUGGA | | GAAACACTAC | |
| xla-miR-142-3p MIMAT0046455 | UGUAGUGUUUCC | 197 | TCCATAAAGTAG | 343 |
| Xenopus laevis miR-142-3p | UACUUUAUGGA | | GAAACACTACA | |
| xtr-miR-142-3p MIMAT0003603 | UGUAGUGUUUCC | 198 | TCCATAAAGTAG | 344 |
| Xenopus tropicalis miR-142-3p | UACUUUAUGGA | | GAAACACTACA | |

In some embodiments, the microRNA target site is such that the minimum free energy or minimum folding energy (MFE) of the microRNA target site bound to a cognate microRNA is less than −35 kcal/mol. Further provided herein are the following specific non-limiting examples of possible microRNA target sites. For example, a nucleic acid molecule which comprises one or more microRNA response elements which correspond to Homo sapien microRNA 106a-3p (hsa-miR-106a-5p) comprising the sequence 5′-CTACCTGCACTGTAAGCACTTTT-3′ (SEQ ID NO: 205) which binds to hsa-miR-106a-5p with an MFE of −44.0 kcal/mol. In another example, the microRNA target site may represent one or more targets corresponding to Homo sapien microRNA 142-3p (hsa-miR-142-3p) comprising the sequence 5′-TCCATAAAGTAGGAAACACTACA-3′ (SEQ ID NO: 213) which would bind to hsa-miR-142-3p with an MFE of −41.4 kcal/mol.

The calculations of MFE are used to predict RNA folding and RNA:RNA interactions. The calculations to define MFE rely on models for Watson-Crick paired helices and many studies that have measured the stability of different nucleic acid-based structures such as hairpin loops, small internal loops, and stacked helices. Based on this data, one can calculate the thermodynamic parameters in a sequence-dependent manner. These algorithms have been developed and used to generate a value for MFE to predict the energetically optimal way in which a miRNA is hybridized to its target. The algorithm forbids intramolecular base pairing and branching structures and utilizes all possible start positions in the miRNA and the target to determine the most optimal MFE. A detailed description of how these calculations are made can be found in Mathews, D. H., et al. Expanded sequence dependence of thermodynamic parameters improves prediction of RNA secondary structure. J Mol Biol 288, 944-940 (1999).

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In some embodiments, complementary oligomeric compounds or regions are complementary at 70% of the nucleobases (70% complementary), 80% complementary, 90% complementary, 95% complementary, or 100% complementary. As used herein, “fully complementary” in reference to an oligonucleotide or portion thereof means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand. As used herein, “non-complementary” in reference to nucleobases means a pair of nucleobases that do not form hydrogen bonds with one another.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases. As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

### II. VECTOR SYSTEMS, CELLS, AND COMPOSITIONS

a. Vector Systems

In some embodiments, the CRISPR-Cas systems describe herein can be delivered to the host cell via one or more vectors, such as viral vectors. For example, the one or more viral vectors may comprise an adenovirus, a lentivirus, adeno-associated virus, or RNA-based viral vectors which may be replication competent or may only encode genes for self-amplification, the later constructs will herein be referred to as replicons.

The term “vector” refers to a nucleic acid molecule capable of transporting another nucleic acid linked thereto. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double-stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication-defective adenoviruses, adeno-associated viruses, and/or RNA-based replicons). Viral vectors also include polynucleotides carried by a virus for transfection into a host cell. Certain vectors are capable of autonomous replication in a host cell into which they are introduced (e.g., RNA vectors comprising their own RNA-dependent RNA polymerase, bacterial vectors having a bacterial origin of replication, and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” is intended to include promoters, enhancers, internal ribosomal entry sites (IRES), and other expression control elements (e.g., transcription termination signals, such as polyadenylation signals and poly-U sequences as well as RNA elements required for recognition by self-encoded RNA dependent RNA polymerases). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

In one aspect, the disclosure provides a vector system or eukaryotic host cell comprising (i) the open reading frame for a Cas member capable of dual RNAse and DNase activity; and (ii) a non-coding RNA sequence comprising in 5′ to 3′ direction encompassing (a) a direct repeat sequence recognizable to the cognate Cas protein (b) a guide nucleotide sequence encoding or comprising a crRNA capable of hybridizing with a target sequence and forming a complex with a Cas protein that has both RNase and DNase activity (c) a second direct repeat sequence recognizable to the cognate Cas protein. In some embodiments, component (c), the second direct repeat can be replaced with a microRNA-target site capable of hybridizing with a microRNA that mediates cleavage of the microRNA-target site by a microRNA-associated protein. In some embodiments, the guide nucleotide sequence further comprises an AU-rich element, a degradation tag, or a combination thereof, located downstream from the microRNA-target site. In some embodiments, the Cas nucleotide sequence and the guide nucleotide sequence are located on the same vector. The Cas nucleotide sequence and the guide nucleotide sequence are located on different vectors.

In some embodiments, the vector system may include one or more viral vectors. In some embodiments, the one or more viral vectors comprise an adenovirus-based vector, a lentivirus-based vector, an adeno-associated virus-based vector, or an RNA-based replicon. In some embodiments, when expressed, the aforementioned CRISPR-Cas system can bind and cleave at the direct repeat sequence, thus preventing functional virion formation. For example, crRNA can be encoded in the 3′-UTR of Cas12a that leads to self-cleavage of its own transcript. As a result, functional virions will not be assembled. Accordingly, the vector system described herein includes a self-replicating RNA (e.g., Nodamurovirus-based replicon) that makes the Cas protein (e.g., Cas12a) and crRNA and simultaneously self-inactivates upon execution of its function.

In yet another aspect, this disclosure provides a polynucleotide molecule comprising a polynucleotide sequence encoding one or more components of a CRISPR-Cas system with both RNase and DNase activity (e.g., Cas12a). In some embodiments, the polynucleotide comprises (i) the open reading frame for a Cas member capable of dual RNAse and DNase activity; and (ii) a non-coding RNA sequence comprising in 5′ to 3′ direction encompassing (a) a direct repeat sequence recognizable to the cognate Cas protein (b) a guide nucleotide sequence encoding or comprising a crRNA capable of hybridizing with a target sequence and forming a complex with the CRISPR-Cas protein (c) at least one microRNA-target site capable of hybridizing with a microRNA that mediates cleavage of the microRNA-target site by a microRNA-associated protein. In some embodiments, the guide nucleotide sequence further comprises an AU-rich element, a degradation tag, or a combination thereof, located downstream from the microRNA-target site.

In some embodiments, the polynucleotide may further comprise one or more regulatory elements which are operably linked to the polynucleotide sequence encoding one or more components of the aforementioned CRISPR-Cas system. The regulatory element may be operably configured for expression of the component(s) of the CRISPR-Cas system with both RNase and DNase activity within in a eukaryotic cell. In some embodiments, the eukaryotic cell may be a human cell, a rodent cell, optionally a mouse cell, a yeast cell, or an insect cell. In some embodiments, the eukaryotic cell may be a Chinese hamster ovary (CHO) cell.

In some embodiments, the CRISPR-Cas systems disclosed herein or the compositions comprising the disclosed Cas12a-based systems may be delivered via liposomes, particles (e.g., nanoparticles), exosomes, microvesicles, a lipid, a cell-penetrating peptide (CPP) or a gene-gun. Delivery vehicles, particles, nanoparticles, formulations, and components thereof for expression of one or more elements of the aforementioned CRISPR-Cas systems are as used in PCT/US2013/074667.

In one aspect, this disclosure provides a composition comprising one or more vectors, liposomes, particles (e.g., nanoparticles, lipid nanoparticles), exosomes, or microvesicles that include one or more components of CRISPR-Cas system with both RNase and DNase activity.

In another aspect, this disclosure provides a host cell or cell line or progeny thereof comprising the aforementioned CRISPR-Cas system, the vector system, or the polynucleotide, as described above. The cell may be a eukaryotic cell (e.g., a plant, animal, or human cell) or a prokaryotic cell. Also provided is a product of any such cell or of any such progeny, resulted from the one or more target loci modified by the CRISPR-Cas system. The product may be a peptide, polypeptide, or protein.

### III. METHODS AND USES

This disclosure also encompasses methods and uses of the CRISPR-Cas systems described herein for modifying a target DNA sequence (e.g., a chromosomal sequence) or target RNA sequence, e.g., for altering or manipulating the expression of one or more genes or the one or more gene products, in prokaryotic or eukaryotic cells, in vitro, in vivo, or ex vivo. The disclosed CRISPR-Cas systems (e.g., Cas12a-based system) provide an effective means for modifying (e.g., deleting, inserting, translocating, inactivating, activating) a target DNA (double-stranded, linear or super-coiled) in a multiplicity of cell types. Thus, the disclosed CRISPR-Cas systems have a broad spectrum of applications in, e.g., gene therapy, drug screening, disease diagnosis, and prognosis.

a. Methods of Modifying Expression of a Target Polynucleotide

In one aspect, the disclosure provides a method of modifying expression of a target polynucleotide (e.g., target sequence of interest) in a eukaryotic cell. In some embodiments, the method allows a CRISPR-Cas complex (e.g., Cas12a/crRNA complex) to bind to the target polynucleotide, resulting in increased or decreased expression of the target polynucleotide or a gene comprising the target polynucleotide. In some embodiments, the CRISPR-Cas complex comprises Cas12a complexed with a crRNA sequence hybridized to a target sequence within the polynucleotide, wherein the crRNA sequence is linked to a direct repeat sequence.

In some embodiments, the modification comprises cleaving one or two strands at the location of the target sequence by the Cas12a protein. In some embodiments, the modification results in decreased or increased transcription of a target gene. In some embodiments, the method further comprises repairing the cleaved target polynucleotide by homologous recombination with an exogenous template polynucleotide, wherein the repair results in a mutation comprising an insertion, deletion, or substitution of one or more nucleotides of the target polynucleotide. In some embodiments, the mutation results in one or more amino acid changes in a protein expressed from a gene comprising the target sequence. In some embodiments, the method further comprises delivering one or more vectors to a host cell (e.g., eukaryotic cell). In some embodiments, the vectors are delivered to the host cell in a subject. In some embodiments, the modification takes place in the eukaryotic cell in cell culture. In some embodiments, the method further comprises isolating the eukaryotic cell from a subject prior to the modification. In some embodiments, the method further comprises returning the eukaryotic cell and/or cells derived therefrom to the subject.

In some embodiments, the method of modifying a target polynucleotide comprises delivering the system, the isolated nucleic acid, or the particle, as described above, to a target sequence or a cell containing the target sequence. In some embodiments, following formation of a complex between the crRNA and the CRISPR-Cas protein and hybridization of the crRNA to one or more nucleic acid of the target sequence, the CRISPR-Cas protein induces a modification (e.g., cleavage) of the target sequence.

The target polynucleotide has no sequence limitation except that the sequence is followed (downstream or 3′) by a PAM sequence, as described above. Other examples of PAM sequences are given above, and the skilled person will be able to identify further PAM sequences for use with a given CRISPR protein. The target polynucleotide can be in the coding region of a gene, in an intron of a gene, in a control region between genes, etc. The gene can be coding or non-coding.

The target polynucleotide can be any polynucleotide endogenous or exogenous to the cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide).

The method further comprises maintaining the cell or embryo under appropriate conditions such that the crRNA guides the Cas protein to the targeted site in the target sequence to modify the target sequence. In general, the cell can be maintained under conditions appropriate for cell growth and/or maintenance. Suitable cell culture conditions are well known in the art and are described, for example, in Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001), Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the proteins and RNA scaffold, if necessary. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into a uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal-derived from the embryo. Such an animal would comprise the modified chromosomal sequence in every cell of the body.

b. Methods of Generating a Model Eukaryotic Cell

In one aspect, this disclosure provides a method of generating a model eukaryotic cell comprising a mutated disease gene, which can be any gene associated with an increase in the risk of having or developing a disease. In some embodiments, the method comprises (a) introducing a CRISPR-Cas system with RNase and DNase activity into a eukaryotic cell; and (b) allowing a CRISPR complex (e.g., Cas12a/crRNA complex) to bind to a target polynucleotide to effect cleavage of the target polynucleotide within the disease gene, wherein the crRNA comprising the sequence that is hybridized to the target sequence within the target polynucleotide, thereby generating a model eukaryotic cell comprising a mutated disease gene.

In some embodiments, the cleavage comprises cleaving one or two strands at the location of the target sequence by the Cas12a protein. In some embodiments, the cleavage results in decreased or increased transcription of a target gene. In some embodiments, the method further comprises repairing the cleaved target polynucleotide by non-homologous end joining (NHEJ)-based gene insertion mechanisms with an exogenous template polynucleotide, wherein the repair results in a mutation comprising an insertion, deletion, or substitution of one or more nucleotides of the target polynucleotide. In some embodiments, the mutation results in one or more amino acid changes in protein expression from a gene comprising the target sequence.

A variety of eukaryotic cells are suitable for use in the method. For example, the cell can be a human cell, a non-human mammalian cell, a non-mammalian vertebrate cell, an invertebrate cell, an insect cell, a plant cell, a yeast cell, or a single-cell eukaryotic organism. A variety of embryos are suitable for use in the method. For example, the embryo can be a 1-cell, 2-cell, or 4-cell human or non-human mammalian embryo. Exemplary mammalian embryos, including one-cell embryos, such as mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells, and others. In exemplary embodiments, the cell is a mammalian cell or the embryo is a mammalian embryo. In some embodiments, the non-human mammal cell may include, but not limited to, primate bovine, ovine, porcine, canine, rodent, Leporidae such as monkey, cow, sheep, pig, dog, rabbit, rat or mouse cell. In some embodiments, the cell may be a non-mammalian eukaryotic cell such as poultry bird (e.g., chicken), vertebrate fish (e.g., salmon) or shellfish (e.g., oyster, clam, lobster, shrimp) cell. In some embodiments, the non-human eukaryote cell is a plant cell. The plant cell may be of a monocot or dicot or of a crop or grain plant such as cassava, corn, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

c. Methods of Developing a Biologically Active Agent

In another aspect, this disclosure provides a method for developing a biologically active agent that modulates a cell signaling event associated with a disease gene, which can be any gene associated with an increase in the risk of having or developing a disease. In some embodiments, the method comprises (a) contacting a test agent with a model cell, as described above; and (b) detecting a change in a readout that is indicative of a reduction or an augmentation of a cell signaling event associated with the mutation in the disease gene, thereby developing the biologically active agent that modulates the cell signaling event associated with the disease gene.

d. Methods of Treatment

The above-described CRISPR-Cas system, one or more polynucleotides, or vector or delivery systems can be used in a therapeutic method of treatment. The therapeutic method of treatment may comprise gene or genome editing, or gene therapy. In one aspect, this disclosure provides a method of treating a subject in need thereof, comprising inducing gene editing by transforming the subject with the polynucleotide or any of the vectors as herein described. In some embodiments, the method comprises inducing transcriptional activation or repression by transforming the subject with the polynucleotide or any of the vectors as herein described.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The term “transformed” as used herein, refers to a cell, tissue, organ, or organism into which a foreign nucleic acid molecule, such as a construct, has been introduced. The introduced nucleic acid molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced nucleic acid molecule. Preferably, the transgenic plant is fertile and capable of transmitting the introduced nucleic acid to progeny through sexual reproduction.

Many devastating human diseases have one common cause: genetic alteration or mutation. The disease-causing mutations in patients are either acquired through inheritance from their parents or are caused by environmental factors. These diseases include, but are not limited to, the following categories. First, some genetic disorders are caused by germline mutations. One example is cystic fibrosis, which is caused by mutations at the CFTR gene inherited from parents. A second suppressor mutation in the mutant CFTR can partially restore the function of CFTR protein in somatic tissues. Other example genetic diseases caused by a point genetic mutation that can be corrected by the disclosed technology include Gaucher's disease, alpha trypsin deficiency disease, sickle cell anemia, to name a few. Second, some diseases, such as chronic viral infectious diseases, are caused by exogenous environmental factors and resulting in genetic alterations. One example is AIDS, which is caused by insertion of the human HIV viral genome into the genome of infected T-cells. Third, some neurodegenerative diseases involve genetic alterations. One example is Huntington's disease, which is caused by expansion of CAG tri-nucleotide in the huntingtin gene of affected patients. Finally, cancers are caused by various somatic mutations accumulated in cancer cells. Therefore, correcting the disease-causing genetic mutations, or functionally correcting the sequence, provides an appealing therapeutic opportunity to treat these diseases.

Somatic genetic editing is an appealing therapeutic strategy for many human diseases. Through precise editing of the target DNA or RNA sequence, the CRISPR-Cas system can correct the mutated genes in genetic disorders, inactivate the viral genome in the infected cells, eliminate the expression of the disease-causing protein in neurodegenerative diseases, or silence the oncogenic protein in cancers. Accordingly, the system and method disclosed in this disclosure can be used in correcting underlying genetic alterations in diseases including the above mentioned genetic disorders, chronic infectious diseases, neurodegenerative diseases, and cancer.

Genetic Diseases

It is estimated that over six thousand genetic diseases are caused by known genetic mutations. Correcting the underlying disease-causing mutations in the pathological tissues/organs can provide alleviation or cure to the diseases. For example, cystic fibrosis affects 1 out of every 3,000 people in the US. It is caused by inheritance of a mutated CFTR gene and 70% of the patients have the same mutation, deletion of a tri-nucleotide leading to a deletion of phenylalanine at position 508 (called Δ Phe 508). Δ Phe 508 leads to the mislocation and degradation of CFTR. The system and method disclosed in this invention can be used to convert a Val 509 residue (GTT) to Phe 509 (TTT) in affected tissues (lung), thereby functionally correcting the Δ Phe 508 mutation. In addition, a second suppressor mutation (such as R553Q or R553M or V510D) in the mutant Δ Phe 508 CFTR can partially restore the function of CFTR protein in somatic tissues.

Chronic Infectious Diseases

The system and method as disclosed can also be used to specifically inactivate any gene in a viral genome that is incorporated into human cells/tissues. For example, the system and method disclosed in this invention allow one to create a stop codon for early termination of translation of the essential viral genes, and thereby remediate or cure the chronic debilitating infectious diseases. For example, current AIDS therapies can reduce viral load, but cannot totally eliminate dormant HIV from positive T cells. The system and method disclosed herein can be used to permanently inactivate one or two essential HIV gene expression in the integrated HIV genome in human T-cells by introducing one or two stop codons. Another example is the hepatitis B virus (HBV). The system and method disclosed here can be used to specifically inactivate one or two essential HBV genes, which are incorporated into the human genome, and silence HBV life-cycle.

Neurodegenerative Diseases

Some neurodegenerative diseases are caused by gain-of-function mutations. For example, SOD1G93A leads to development of amyotrophic lateral sclerosis (ALS). The system and method disclosed in this invention can be used to either correct the mutation or eliminate the mutant protein expression by introducing a stop codon or by changing a splicing site.

Diseases of the Muscular System

The present invention also contemplates delivering the CRISPR-Cas system described herein to muscle(s). Dystrophin is a cytoplasmic protein that provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids. Exon 51 is frequently adjacent to frame-disrupting deletions in DMD patients and has been targeted in clinical trials for oligonucleotide-based exon skipping. A clinical trial for the exon 51 skipping compound eteplirsen recently reported a significant functional benefit across 48 weeks, with an average of 47% dystrophin positive fibers compared to baseline. Mutations in exon 51 are ideally suited for permanent correction by NHEJ-based genome editing. The methods of US Patent Publication No. 20130145487, which relates to meganuclease variants to cleave a target sequence from the human dystrophin gene (DMD), may also be modified for the nucleic acid-targeting system of the present invention.

Cancers

Many genes (including tumor suppressor genes, oncogenes, and DNA repair genes) contribute to the development of cancer. Mutations in these genes often lead to various cancers. Using the system and method disclosed herein, one can specifically target and correct these mutations. As a result, causative oncogenic proteins can be functionally annulled or their expression can be eliminated by introducing a point mutation at either the catalytic sites or splicing sites. In some embodiments, the treatment, prophylaxis or diagnosis of cancer is provided. The target is preferably one or more of the FAS, BID, CTLA4, PDCD1, CBLB, PTPN6, TRAC, or TRBC genes. Cancer may be one or more of lymphoma, chronic lymphocytic leukemia (CLL), B cell acute lymphocytic leukemia (B-ALL), acute lymphoblastic leukemia, acute myeloid leukemia, non-Hodgkin's lymphoma (NHL), diffuse large cell lymphoma (DLCL), multiple myeloma, renal cell carcinoma (RCC), neuroblastoma, colorectal cancer, breast cancer, ovarian cancer, melanoma, sarcoma, prostate cancer, lung cancer, esophageal cancer, hepatocellular carcinoma, pancreatic cancer, astrocytoma, mesothelioma, head and neck cancer, and medulloblastoma. This may be implemented with engineered chimeric antigen receptor (CAR) T cell. This is described in WO2015161276, the disclosure of which is hereby incorporated by reference and described hereinbelow. Target genes suitable for the treatment or prophylaxis of cancer may include, in some embodiments, those described in WO2015048577 the disclosure of which is hereby incorporated by reference.

Stem Cell Genetic Modification

In some embodiments, stem cell or progenitor cell can be genetically modified using the system and method disclosed in this invention. Suitable cells include, e.g., stem cells (adult stem cells, embryonic stem cells, iPS cells, etc.) and progenitor cells (e.g., cardiac progenitor cells, neural progenitor cells, etc.). Suitable cells include mammalian stem cells and progenitor cells, including, e.g., rodent stem cells, rodent progenitor cells, human stem cells, human progenitor cells, etc. Suitable host cells include in vitro host cells, e.g., isolated host cells.

In some embodiments, the present invention can be used for targeted and precise genetic modification of tissue ex vivo, correcting the underlying genetic defects. After the ex vivo correction, the tissues may be returned to the patients. Moreover, the technology can be broadly used in cell-based therapies for correcting genetic diseases.

Genetic Editing in Animals and Plants

The system and method described above can be used to generate a transgenic non-human animal or plant having one or more genetic modification of interest. In some embodiments, the transgenic non-human animal is homozygous for the genetic modification. In some embodiments, the transgenic non-human animal is heterozygous for the genetic modification. In some embodiments, the transgenic non-human animal is a vertebrate, for example, a fish (e.g., zebrafish, goldfish, pufferfish, cavefish, etc.), an amphibian (frog, salamander, etc.), a bird (e.g., chicken, turkey, etc.), a reptile (e.g., snake, lizard, etc.), a mammal (e.g., an ungulate, e.g., a pig, a cow, a goat, a sheep, etc.; a lagomorph (e.g., a rabbit); a rodent (e.g., a rat, a mouse); or a non-human primate.

The invention can be used for treating diseases in animals in a way similar to those for treating diseases in humans as described above. Alternatively, it can be used to generate knock-in animal disease models bearing specific genetic mutation(s) for purposes of research, drug discovery, and target validation. The system and method described above can also be used for introduction of point mutations to ES cells or embryos of various organisms, for the purpose of breeding and improving animal stocks and crop quality.

Methods of introducing exogenous nucleic acids into plant cells are well known in the art. Suitable methods include viral infection (such as double-stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e. in vitro, ex vivo, or in vivo).

e. Kits

This disclosure further provides kits containing reagents for performing the above-described methods, including CRISPR:Cas guided target binding or correction reaction. To that end, one or more of the reaction components, e.g., RNAs, Cas proteins, and related nucleic acids, for the methods disclosed herein can be supplied in the form of a kit for use. In one embodiment, the kit comprises a CRISPR protein or a nucleic acid encoding the Cas protein, effector protein, one or more of an RNA scaffold described above, a set of RNA molecules described above. In some embodiments, the kit can include one or more other reaction components. In such a kit, an appropriate amount of one or more reaction components is provided in one or more containers or held on a substrate.

Examples of additional components of the kits include, but are not limited to, one or more host cells, one or more reagents for introducing foreign nucleotide sequences into host cells, one or more reagents (e.g., probes or PCR primers) for detecting expression of the RNA or protein or verifying the target nucleic acid's status, and buffers or culture media for the reactions (in 1× or concentrated forms). The kit may also include one or more of the following components: supports, terminating, modifying or digestion reagents, osmolytes, and an apparatus for detection.

The reaction components used can be provided in a variety of forms. For example, the components (e.g., enzymes, RNAs, probes, and/or primers) can be suspended in an aqueous solution or as a freeze-dried or lyophilized powder, pellet, or bead. In the latter case, the components, when reconstituted, form a complete mixture of components for use in an assay.

The kits of the invention can be provided at any suitable temperature. For example, for storage of kits containing protein components or complexes thereof in a liquid, it is preferred that they are provided and maintained below 0° C., preferably at or below −20° C., or otherwise in a frozen state.

A kit or system may contain, in an amount sufficient for at least one assay, any combination of the components described herein. In some applications, one or more reaction components may be provided in pre-measured single-use amounts in individual, typically disposable, tubes or equivalent containers. With such an arrangement, an RNA-guided reaction can be performed by adding a target nucleic acid, or a sample or cell containing the target nucleic acid, to the individual tubes directly. The amount of a component supplied in the kit can be any appropriate amount and may depend on the target market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for the use of the components.

### IV. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The terms “polypeptide,” “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product(s).” If the polynucleotide is derived from genomic DNA, expression may include splicing of the mRNA in a eukaryotic cell.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas12a polynucleotides are derived from the wild type Cas12a protein amino acid sequence. Also, the variant mammalian codon-optimized Cas12a polynucleotides, including the Cas12a single mutant nickase and Cas12a double mutant null-nuclease, are derived from the polynucleotide encoding the wild type mammalian codon-optimized Cas12a protein.

As used herein the term “wild type” is a term of the art understood by skilled persons and means the typical form of an organism, strain, gene, or characteristic as it occurs in nature as distinguished from mutant or variant forms.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. For example, the mutant forms of mammalian codon-optimized Cas12a, including the Cas12a single mutant nickase and the Cas12a double mutant null-nuclease, are variants of the mammalian codon-optimized wild type Cas12a. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial, or inconsequential changes to the parent amino acid sequence. For example, minor, trivial, or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made. Advantageous embodiments can involve engineered or non-naturally occurring Cas proteins having both an RNAse and DNase activity, e.g., Cas12a, or an ortholog or homolog thereof.

The term “isolated” when referring to nucleic acid molecules or polypeptides means that the nucleic acid molecule or the polypeptide is substantially free from at least one other component with which it is associated or found together in nature.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

As used herein, the term “guide RNA” generally refers to an RNA molecule (or a group of RNA molecules collectively) that can bind to a CRISPR protein and target the CRISPR protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence.

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to or modify using a CRISPR system. A target sequence may be within a nucleic acid in vitro or in vivo within the genome of a cell, which may be any form of single-stranded or double-stranded nucleic acid.

A “target nucleic acid strand” refers to a strand of a target nucleic acid that is subject to base-pairing with a crRNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and, thus, are still capable of nucleobase complementarity.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques in Biochemistry and Molecular Biology-Hybridization with Nucleic Acid Probes Part I, Second Chapter “Overview of principles of hybridization and the strategy of nucleic acid probe assay,” Elsevier, N.Y.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, the term “contacting,” when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as an Cas protein or guide RNA (or crRNA), includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution or a number of different elements attached to a solid support at random or in no particular order in which the different elements are not spatially distinct. In other words, a mixture is not addressable.

The term “progeny”, such as the progeny of a transgenic plant, is one that is born of, begotten by, or derived from a plant or the transgenic plant. The introduced nucleic acid molecule may also be transiently introduced into the recipient cell such that the introduced nucleic acid molecule is not inherited by subsequent progeny and thus not considered “transgenic.” Accordingly, as used herein, a “non-transgenic” plant or plant cell is a plant which does not contain a foreign nucleic acid stably integrated into its genome.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “modulate” is meant to refer to any change in biological state, i.e., increasing, decreasing, and the like.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the invention, and are physiologically acceptable to the subject. Supplementary active compounds may also be incorporated into the compositions.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### V. EXAMPLES

### Example 1

This example describes the materials and methods used in EXAMPLES 2-6 below.

### Plasmids

Cas12a plasmids were generated from a synthetic codon-optimized gene derived from Acidaminococcus (SEQ ID NO: 4). For miRNA-mediated enabling of the CRISPR-Cas system, either single or two microRNA response elements corresponded to Homo sapiens microRNA 106a-3p (hsa-miR-106a-5p: comprising the sequence 5′-CTACCTGCACTGTAAGCACTTTT-3′) (SEQ ID NO: 205) or Homo sapiens microRNA 142-3p (hsa-miR-142-3p: comprising the sequence 5′ TCCATAAAGTAGGAAACACTACA-3′) (SEQ ID NO: 213) was introduced. These target sites, the crRNAs flanked by direct repeats (DR), modified direct repeats (A18G, UUAA, scrbl), and the AU-rich element (ARE) described herein were ordered as dsDNA fragments (IDT) and inserted into the 3′UTR of Cas12a using In-Fusion cloning (TAKARA).

### Cells

Fibroblasts used for all experiments were maintained in Dulbecco's Modified Eagle Medium (GIBCO) supplemented with 1× penicillin-streptavidin solution (CORNING) and 10% fetal bovine serum (CORNING).

### Western Blot

Whole-cell extract was prepared from live cells lysed in 1% NP-40 lysis buffer. Protein levels were analyzed by SDS-PAGE on a 4-15% acrylamide gradient gel (BIO-RAD). Gels were transferred onto a 0.45 μm nitrocellulose membrane (BIO-RAD) and blocked in 5% milk in TBST for 1 h at room temperature. Membranes were probed with the following primary antibodies in 5% milk in TBST overnight at 4° C.: anti-HA (clone HA-7, MILLIPORESIGMA), anti-GFP (ab290, ABCAM), anti-IFIT1, (clone D2X9Z, CELLSIGNALING), anti-actin (clone Ab-5, THERMO SCIENTIFIC) and anti-GAPDH (G9545, MILLIPORESIGMA). After 4×5 min washes in 1×TBST, blots were probed with HRP-linked secondary antibody for 1 h at room temperature (anti-mouse, NA931V or anti-rabbit, NA934V, GE HEALTHCARE) and developed using the Immobilon Western HRP Substrate Kit (MILLIPORESIGMA).

### Small RNA Northern Blot

Total RNA was extracted from live cells using TRIzol (INVITROGEN). Northern blot was performed as described in (Pall, G. S. and Hamilton, A. J. Nat Protoc 3, 1077-1084 (2008)) with 20 μg total RNA per sample. Probes included the following: B2M-crRNA (5′-GCTGGATAGCCTCCAGGCCA-3′) (SEQ ID NO: 345), miR-106a (5′-CTACCTGCACTGTAAGCACTTTT-3′) (SEQ ID NO: 205), and U6 (5′-GCCATGCTAATCTTCTCTGTATC-3′) (SEQ ID NO: 346). Probes were labeled with ATP-P32 using T4 polynucleotide kinase (NEB), and blot was exposed to a phosphor screen (GE) and developed on a Typhoon Storage Phosphorimager.

### Flow Cytometry

Roughly 7.5×10⁵ cells/well were plated on 6-well plates. After attaching overnight, cells were transfected using lipofectamine 2000 (Invitrogen) and were passaged 1:5 when they reached ˜80% confluency for up to ten days. For flow cytometry analysis, cells were trypsinized, washed, and stained using the BD Cytofix/Cytoperm Fixation/Permeabilization Kit as per the manufacture's instructions (BD BIOSCIENCES). The following antibodies and dyes were used: anti-human HLA-A,B,C Pacific Blue (clone W6/32, BIOLEGEND), anti-HA Alexa Fluor 647 (clone HA.11, BIOLEGEND), and LIVE/DEAD stain Aqua (THERMOFISHER). Fixed cells were analyzed on a 2019 Attune N×T Flow Cytometer. Data processing was performed with FLOWJO v. 10.6.

### Example 2

In an effort to generate an RNA-based DNA editor that functions in a cell-specific manner that would be amenable for in vivo use, this disclosure combined CRISPR-Cas and miRNA biology. In brief, it utilized the fact that Cas12a processes its own pre-crRNA to make a vector that delivers both Cas12a and crRNA and in doing so, inactivates the vector itself. To this end, crRNAs was encoded in the 3′-UTR of Cas12a, and it was shown that it leads to self-cleavage of its own transcript. Moreover, this disclosure demonstrated that delivery of this self-inactivating construct is sufficient to achieve efficient gene editing. This disclosure further demonstrated that processing of the pre-crRNA can be made to be dependent on miRNA-expression thereby conferring cell-type specificity on the editing platform.

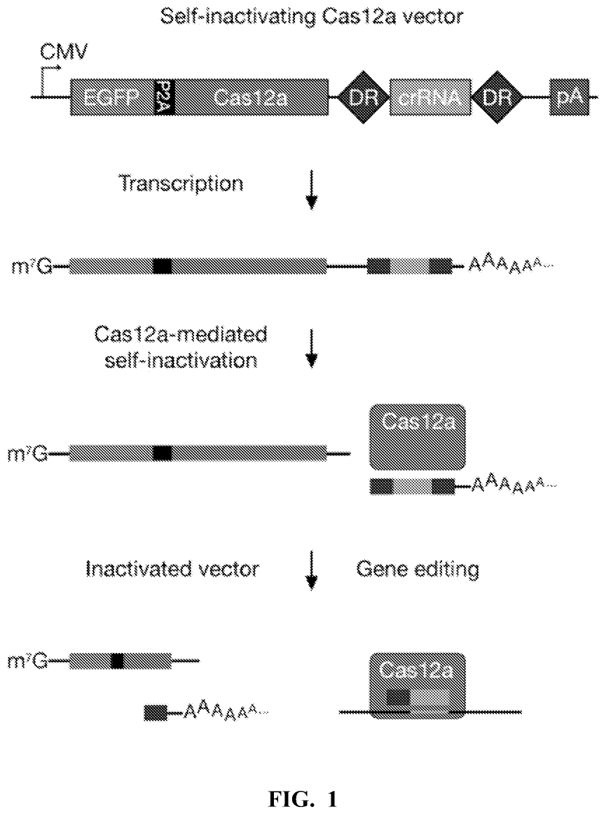

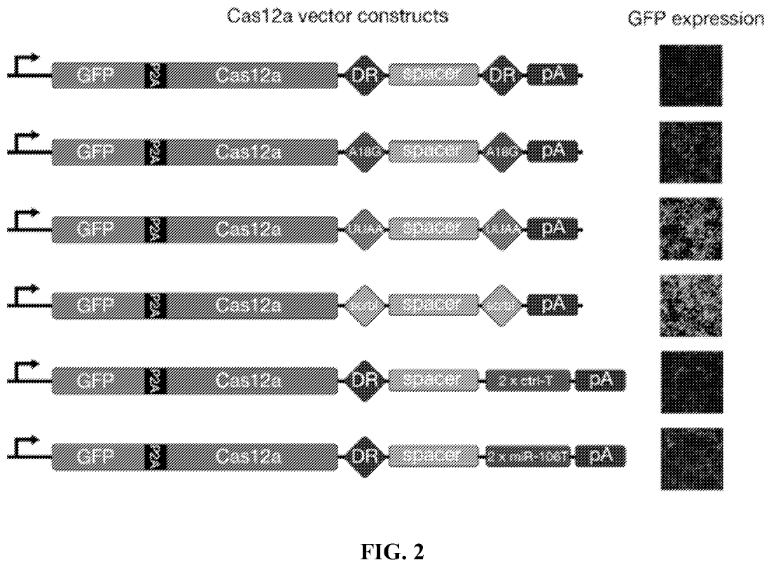

To ascertain whether self-inactivation of Cas12a on a single mRNA transcript can be achieved, a construct encoding an enhanced green fluorescent protein (EGFP) and an HA epitope-tagged Cas12a separated by a P2A peptide site was first generated (Sharma, P. et al. Nucleic Acids Res 40, 3143-3151 (2012)). (FIG. 2) To achieve self-inactivation, a crRNA that targets beta-2 microglobulin (B2M) was further cloned into the 3′ UTR flanked by Cas12a-compatible direct repeats, comprised of a 19 hairpin binding site for the Cas12a nuclease (FIG. 1). Moreover, in an attempt to impact the efficiency in which the crRNA is processed, either canonical direct repeats, direct repeats that would be poorly or unable to be cleaved by Cas12a (A18G and UUAA, respectively), and one in which the direct repeats were disrupted altogether (scrambled; scrbl) were utilized (Zetsche, B. et al. Nat Biotechnol 35, 31-34 (2017); Zhong, G., et al. Nat Chem Biol 13, 839-841 (2017)).

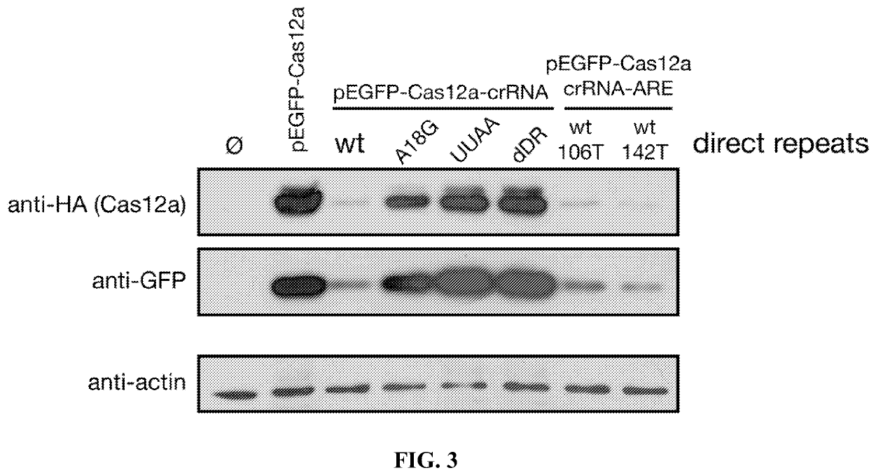

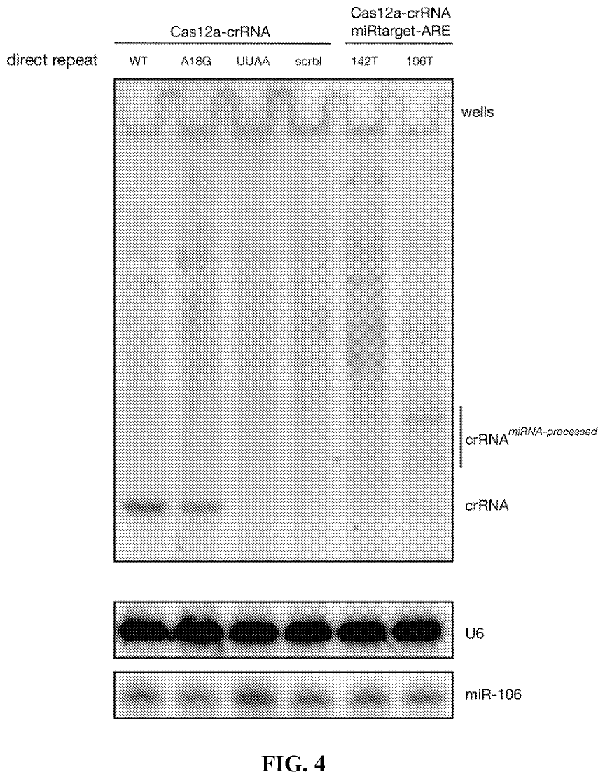

To determine how these constructs would function, they were introduced into fibroblasts and monitored for EGFP expression by both fluorescence microscopy and western blot (FIGS. 2-3). These data demonstrated that the EGFP expression from the construct containing canonical direct repeats showed only low levels of fluorescence or expression by western blot which could also be correlated with HA-Cas12a expression (FIG. 3). When the direct repeats were comprised of the A18G sites, fluorescence increased as compared to canonical sites (FIG. 2). This enhanced expression could also be further corroborated by western blot analysis of both EGFP and HA-Cas12a suggesting self-inactivation was diminished with the A18G sites (FIG. 3). When the direct repeats were made to be uncleavable by Cas12a (UUAA), EGFP expression was comparable to a construct lacking any direct repeats (scrbl) (FIGS. 2-3). As these data indicate, the construct was undergoing Cas12a-mediated self-attenuation. Next, these vectors were introduced into fibroblasts but RNA processing was analyzed by small RNA northern blot (FIG. 4). In agreement with the microscopy and western findings, these data demonstrated that the B2M crRNA derived from the 3′ UTR was generated in a manner inversely proportional to EGFP or HA-Cas12a expression (FIG. 4).

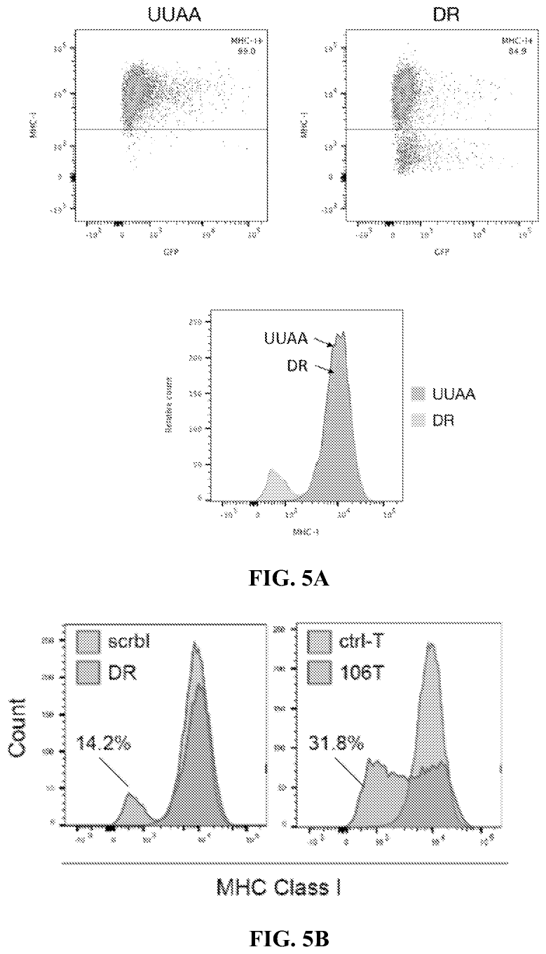

Further, the ability of a construct comprising Cas12a and a B2M-specific crRNA flanked by direct repeats or repeats with nucleotides 16-19 changed from AAUU to UUAA to successfully reduce B2M-dependent expression of MHC-I was assessed by flow cytometry (FIG. 5A). These data revealed a population of cells with disrupted expression indicating editing efficiency to be ˜20% five days post transfection (FIG. 5A).

### Example 3

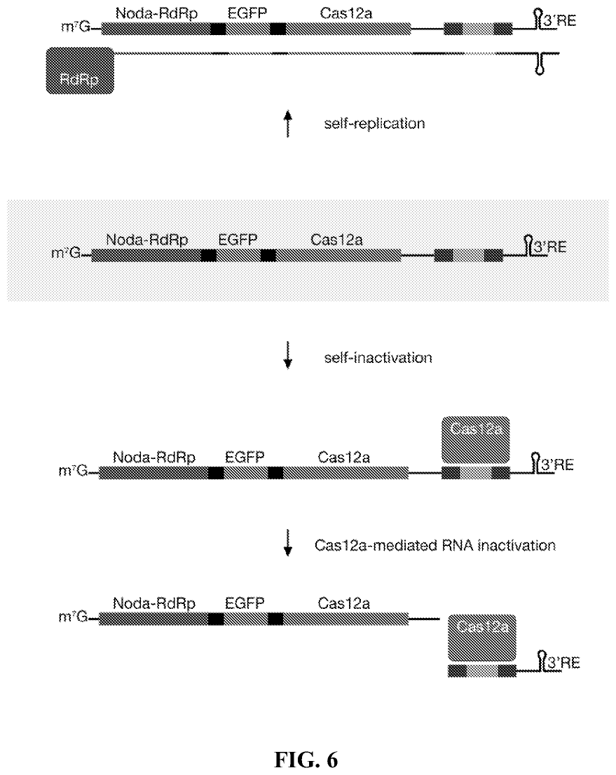

Given the capacity of a single transcript to both yield a functioning Cas12a editing platform and undergo self-inactivation, whether this biologic circuit could be applied to other modalities was assessed. To this end, three of these constructs encoding EGFP-2A-HA-Cas12a and harboring a 3′UTR containing a crRNA targeting B2M flanked by direct repeats that were either canonical, carrying the A18G mutation rendering cleavage suboptimal, or carrying the UUAA that fully abrogates cleavage into the genome of an arthropod virus (FIG. 6) were grafted. Utilizing only the RNA-dependent RNA polymerase (RdRp) of Nodamura virus and the 5′ and 3′ noncoding material required for RdRp recognition, a self-replicating RNA (herein referred to as a replicon) was generated.

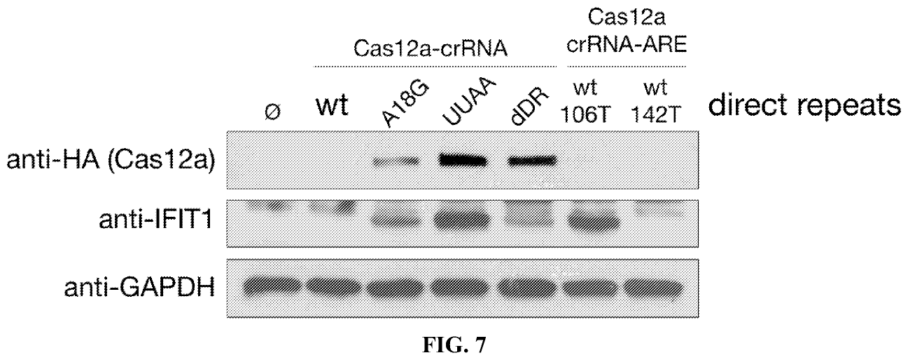

Consistent with the single mRNA transcript data, HA-Cas12a expression was undetectable with the canonical direct repeat, intermediate with A18G repeats, and the highest with HA-Cas12a transcript containing the UUAA motif (FIG. 7). Taken altogether, these data demonstrate that genetic editing can be achieved as a single RNA transcript that also undergoes self-editing to mitigate any risks associated with long term expression. Moreover, as it was shown that this biology can be recapitulated as an RNA with no DNA phase, and in the context of a virus-based vector, this platform is amenable to in vivo utilization.

### Example 4

In this era of synthetic biology, the use of RNA replicons as a therapeutic modality is gaining traction in the scientific community (Lundstrom, K. Genes (Basel) 10 (2019)). However, despite the attractive nature of having a programmable RNA as a delivery vehicle for gene editing in vivo, the nature of a self-amplifying foreign RNA is also likely to engage the host antiviral defenses. This is evident from the UUAA Nodamura virus construct, which results in robust induction of the interferon response as measured by interferon-induced with tetratricopeptide repeats 1 (IFIT1) protein levels (FIG. 7). However, it was found herein that the same biological circuit designed to ensure temporal expression of the genetic editor also ensures that viral pathogen-associated molecular patterns (PAMPs), such as dsRNA, do not accumulate and therefore yield no transcriptional response by the cell as noted by the absence of IFIT1 with direct repeats are canonical (FIG. 7). An intermediate phenotype with the use of the A18G construct was observed (FIG. 7).

### Example 5

In addition to minimizing any unwanted response to the RNA construct, cell specificity is also an important attribute to limiting off-target effects and mitigate overall risk. While the use of receptors from different human pathogens grants some level of tissue tropism, application of many of these constructs is confounded by seroprevalence in the human population. Therefore, it would be preferable to be able to package replicons with a relative promiscuous viral binding protein with little to no seroprevalence in the human population. A great example of this would be the use of the glycoprotein G of vesicular stomatitis virus which has already been shown to be compatible with replicon biology (Zetsche, B. et al. Nat Biotechnol 35, 31-34 (2017)). However, if entry is ubiquitous, most vectors must gain specificity through the use of cell type-specific promoters—an attribute only applicable to DNA-based delivery systems. In an effort to achieve this in the absence of DNA, host miRNA targeting and cleavage was exploited which functions at the level of RNA. Given the known specificity for which miRNAs can be made to cut, miRNA biology was harnessed to further control the RNA-based editor. To this end, cell-specific miRNAs were used, which have been identified through numerous small RNA sequencing efforts (Landgraf, P. et al. Cell 129, 1401-1414 (2007)), and the 3′ canonical direct repeat was replaced with miRNA targets corresponding to either a ubiquitous miRNA (miR-106a) or one which is confined to the hematopoietic lineage and absent in fibroblasts (miR-142-3p) (FIG. 8) (Meier, J. et al. RNA Biol 10, 1018-1029 (2013); Chen, C. Z., et al. Science 303, 83-86 (2004)). In the presence of the cognate miRNA, Ago2 as part of the RNA induced silencing complex (RISC), will be recruited and result in 3′ cleavage of the crRNA. As miRNAs can be cell-specific, this synthetic construct would inactivate itself ubiquitously while only generating functional crRNA in a desired cell type where the cognate miRNA is present. Moreover, while this construct would still self-inactivate in the presence of only a single direct repeat, an RNA destabilizing element (ARE) was further added on the 3′UTR to ensure rapid RNA turnover in the absence of processing of the 3′ side of the crRNA (Younis, I. et al. Mol Cell Biol 30, 1718-1728 (2010)).

To characterize the behavior of this Cas12a/miRNA-based genetic editor, the genetic editor was introduced into fibroblasts to ascertain how the design of different 3′UTRs would impact HA-Cas12a or EGFP expression (FIG. 3). As previously demonstrated, introduction of an RNA encoding EGFP-2A-Cas12a with either no UTR, a UTR lacking direct repeats, or one in which the direct repeats are uncleavable (UUAA) yielded robust Cas12a and EGFP expression (FIG. 3). As previously observed, flanking the B2M crRNA in the 3′UTR with canonical direct repeats led to near undetectable levels of Cas12a and a significant loss of EGFP signal, and when the B2M crRNA was flanked with the mutated direct repeat (A18G), intermediate levels of EGFP and Cas12a were achieved. In contrast, when the 3′ direct repeat was replaced with miRNA target sites for either miR-106a (expressed in fibroblasts) or miR-142-3p (an irrelevant control target sequence (ctrl-T), absent in fibroblasts), levels of Cas12a and EGFP that were comparable to the wild type self-targeting construct. The wild-type direct repeat on the 5′ end had been kept to mediate self-inactivation. These data suggest that a single direct repeat is sufficient for self-inactivation, although it is noteworthy that those transcripts that escape Cas12a-cleavage are being processed by miR-106a as levels of both EGFP and Cas12a are more elevated when compared to the miR142T (control) construct, presumably due to the loss of the ARE destabilizing element (FIG. 3).

### Example 6

To further characterize the behavior of this genetic design, these same constructs by small RNA northern blot were also evaluated (FIG. 4). In agreement with the expression data for Cas12a and EGFP, an inverse correlation between Cas12a and crRNA levels with abundant B2M-specific crRNA found in the construct containing canonical direct repeats was observed. Levels of crRNA were again intermediate for A18G sites, and undetectable for UUAA or B2M crRNAs flanked by scrambled sequences. In contrast, replacing the 3′ canonical direct repeat with either miR-106a or miR-142-3p (control) target sites showed only crRNA in response to incorporation of miR-106a target sites: The crRNA is no longer processed when the 3′ direct repeat is replaced with the control miRNA target sequence, indicating a lack of cleavage (FIG. 4). Note that the 3′ extension that accounts for the crRNAs increase in size represents the additional 10 nucleotides remaining from the cleavage of the miRNA target site.

To ascertain whether the product of 5′ direct repeat and a 3′ miRNA cleavage site remains functional, variants of the RNA construct that encoded a crRNA targeting beta 2 microglobulin (B2M) were expressed. In comparing transcripts lacking direct repeats (scrbl), having both direct repeats, or containing a 5′ direct repeat with either a control 3′ target sequence (miR-142-3p) or miR-106a 3′ sites, loss of MHC Class I, a proxy for B2M targeting, was observed only in conditions in which the 3′ end of the spacer contained a wild type direct repeat or the miR-106a target sites (FIG. 4). These data demonstrate a ˜14% reduction of MHC1 with the canonical Cas12a targeting system which increases to greater than 30% targeting in the presence of miR-106a despite the extended crRNA (FIGS. 4 and 5B). In contrast the miR-142-3p (ctrl-T) construct showed no editing in the absence of this hematopoietic-specific miRNA (FIG. 5B).

Together, these data suggest that miRNA biology can be exploited in conjunction with Cas12a-based processing to generate a single RNA capable of both self-inactivation and cell-specific targeting.

### Example 7

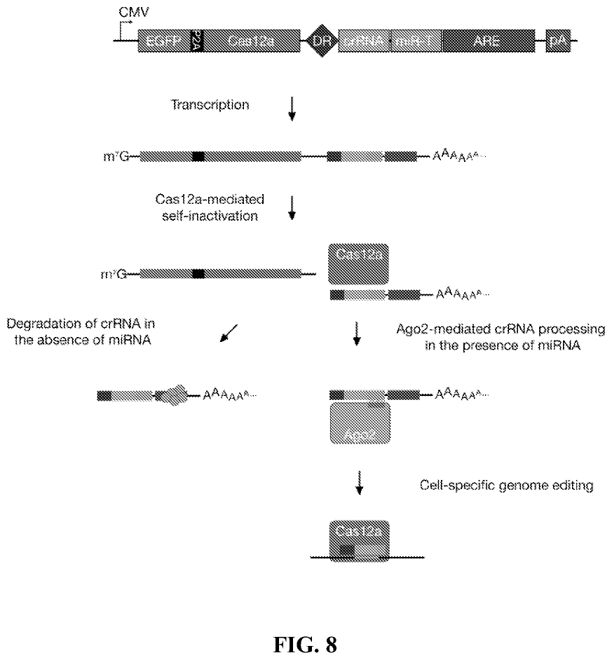

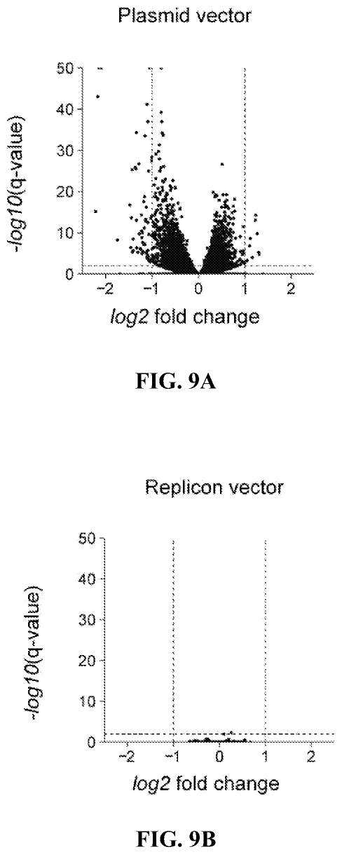

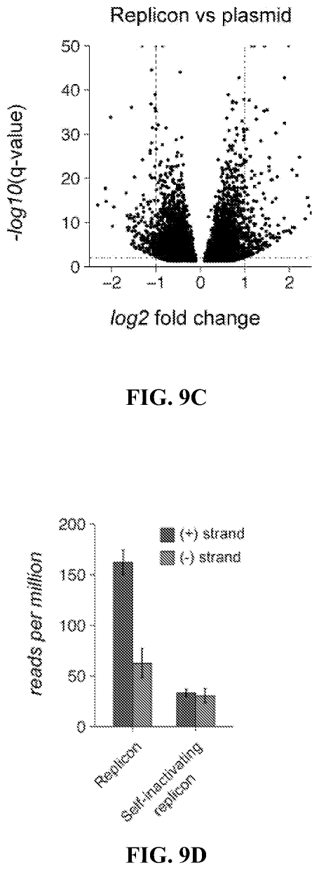

To determine whether the transcriptional response to the self-inactivating constructs would be amenable to in vivo use, bulk RNA sequencing was performed to ascertain the transcriptional response to Cas12a expression and/or crRNA processing. To this end, the expression of Cas12a that was capable of self-inactivation was compared the expression of Cas12a that was incapable of self-inactivation. The sequencing data set revealed that in contrast to sustained expression of Cas12a alone, the self-inactivating plasmid resulted in a significant number of differentially expressed genes (DEGs) (FIG. 9A). All upregulated genes with a log 2fold change greater than 1 and an adjusted p-value less than 0.01 were annotated as belonging to the interferon response. These data would indicated that Cas12a processing of its own RNA results in a significant accumulation of aberrant RNA capable of inducing the host antiviral defenses. In contrast, the same comparison using the replicon-based platform yielded no DEGs (FIG. 9B). To determine if the lack of an interferon signature in response to the replicon-based platform was simply the result of having it generated in both conditions as a result of RdRp activity, the plasmid-based Cas12a system was compared with processable crRNA to the equivalent replicon platform (FIG. 9C). This comparison yielded a larger number of DEGs, but the interferon signature remained limited to plasmid-based delivery of Cas12a and crRNA, demonstrating that the replicon self-inactivation is potent enough to prevent a cellular antiviral response. This was further corroborated by replicon read numbers which show that self-inactivation prevents any accumulation of either positive or negative sense transcripts that might otherwise serve as pathogen associated molecular patterns (FIG. 9D).

Here, data demonstrating that RNA-based platforms were designed to support safe, efficient, and cell-specific genetic editing have been presented. Based on the dual RNase and DNase properties of Cas12a, it was shown that RNA constructs can be engineered to be self-targeting. This attribute not only ensures that Cas12a and crRNA expression is temporal, thereby minimizing off-target editing, but it also keeps foreign RNA levels below the cellular threshold for which interferon and the antiviral defenses are induced. This could be observed with the correlation between Cas12a expression and that of IFIT1—a canonical interferon-stimulated response gene. Taken together, these results demonstrate that the use of RNA-only vectors to engineer genetic editors is both feasible and safe.

A remaining attribute that diminishes the full potential of RNA- or replicon-based therapeutics is the difficulty in achieving specificity. Historically, nucleic acid-based therapeutics and gene therapy vectors relied on promoter elements that were uniquely specific to a desired cell type. While this strategy has achieved some noteworthy successes, use of DNA as a vector introduces other unwanted issues including the need for entry into the nucleus and the possibility of genomic integration. RNA-based vectors mitigate this risk by having no DNA phase and performing all of their functions in the cytoplasm. Given these attributes, miRNA-based targeting was adapted as a means of instilling cell-specific activity. Here, it was shown that the addition of a perfect complementary miRNA can replace the 3′ direct repeat needed to liberate a desired crRNA.

Furthermore, it was demonstrated that this same system could be coupled with destabilizing elements to further control the extent of self-targeting and clearance of any incoming material. Lastly, while rapid turnover is appealing as a means of mitigating the risk associated with off-target effects, it cannot be conferred at the cost of targeting efficiency. For this reason, the miR-106a and miR-142-3p targeted constructs were tested in the presence of only miR-106a expression, and it was found that it could mediate its activity in a manner that was miRNA-specific. Together with the knowledge that every tissue or cell-type has a unique miRNA profile, these data demonstrate that one can engineer an RNA-based vector to efficiently enter the cytoplasm and then function only in those cells where editing is desired.

1. A system for microRNA-enabled gene editing, comprising:
(i) a Cas nucleotide sequence encoding a CRISPR-Cas protein with both RNAse and DNase activity; and
(ii) a targeting sequence comprising in 5′ to 3′ direction
(a) a direct repeat sequence,
(b) a guide nucleotide sequence encoding or comprising a crRNA sequence capable of hybridizing with a target sequence and forming a complex with the CRISPR-Cas protein, and
(c) at least one microRNA target site capable of hybridizing with a microRNA that mediates cleavage of the microRNA-target site by a microRNA-associated protein.

2. The system of claim 1, wherein the system is a nucleic acid.

3. The system of claim 2, wherein the system is an RNA.

4. The system of claim 3, wherein the guide nucleotide sequence further comprises an AU-rich element, a degradation tag, or a combination thereof, located downstream from the microRNA-target site.

5. The system of claim 1, wherein the Cas nucleotide sequence and the guide nucleotide sequence are located on a same vector.

6. The system of claim 1, wherein the Cas nucleotide sequence and the guide nucleotide sequence are located on different vectors.

7. The system of claim 1, wherein the microRNA-target site is selected from the group consisting of SEQ ID NOs: 199-344.

8. The system of claim 1, wherein the microRNA-associated protein is Argonaute 2 (Ago2).

9. The system of claim 1, wherein when the crRNA sequence forms a complex with the CRISPR-Cas protein and hybridizes to the target sequence, the CRISPR-Cas protein induces distal cleavage of the target sequence.

10. The system of claim 1, wherein the CRISPR-Cas protein is a Cas12a protein.

11. The system of claim 10, wherein the Cas12a protein is derived from a bacterial species selected from the group consisting of Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae.

12. The system of claim 10, wherein the Cas12a protein is PaCpf1p, LbCpf1, or AsCpf1.

13. The system of claim 10, wherein the Cas12a protein has at least 75% sequence identity with SEQ ID NOs: 1-19.

14. The system of claim 10, wherein the Cas12a protein comprises one or more nuclear localization signals.

15. The system of claim 1, wherein the crRNA sequence is 20-30 nucleotides in length.

16. The system of claim 1, wherein the target sequence is within a cell.

17. The system of claim 1, wherein the target sequence comprises DNA.

18. A host cell or cell line or progeny thereof comprising the system of claim 1.

19. The host cell or cell line or progeny thereof of claim 18, comprising a stem cell or stem cell line.

20. A composition comprising the system of claim 1.

21. A method of modifying a target sequence of interest comprising delivering the system of claim 1 to the target sequence or a cell containing the target sequence.

22. The method of claim 21, wherein following formation of a complex between the crRNA sequence and the CRISPR-Cas protein and hybridization of the crRNA sequence to one or more nucleic acid of the target sequence, the CRISPR-Cas protein induces a modification of the target sequence.

23. The method of claim 21 or 22, wherein the target sequence is located at genomic loci of interest.

24. The method of claim 21, wherein the target sequence comprises DNA.

25. The method of claim 24, wherein the DNA is relaxed or supercoiled.

26. The method of claim 21, wherein the system or the isolated nucleic acid is delivered via particles, vesicles, or one or more viral vectors.

27. The method of claim 26, wherein the one or more viral vectors comprise an adenovirus-based vector, a lentivirus-based vector, or an adeno-associated virus-based vector.

28. The method of claim 21, wherein the modification of the target sequence is a strand break.

29. The method of claim 28, wherein the target sequence is modified by the integration of a DNA insert into the staggered DNA double-stranded break.

30. The method of claim 21, wherein the target sequence is associated with a disease.

31. The method of claim 30, wherein the disease is caused by a genetic defect in the target sequence.

32. The method of claim 30, wherein the disease is cancer.

33. The system of claim 16, wherein the cell is a eukaryotic cell.

34. The system of claim 16, wherein the cell is a plant, animal, or human cell.
